(19) United States
(12) Patent Application Publication
(10) Pub. No.: US 2024/0058437 A1
(43) Pub. Date: Feb. 22, 2024

(54) IMMUNOGENIC PEPTIDE

(71) Applicant: UCL BUSINESS LTD., London (GB)

(72) Inventors: Matthew Bryan Reeves, London (GB); Ilona Anna Baraniak, London (GB); Paul David Griffiths, London (GB)

(21) Appl. No.: 18/267,241

(22) PCT Filed: Dec. 17, 2021
(86) PCT No.: PCT/GB2021/053351

§ 371 Date: Jun. 14, 2023

(30) Foreign Application Priority Data
Dec. 18, 2020 (GB) 2020135.6

(51) Int. Cl. A61K 39/245; C07K 14/045; C07K 16/08; A61K 45/06; A61P 31/22

(52) U.S. Cl. CPC A61K 39/245; C07K 14/045; C07K 16/089; A61K 45/06; A61P 31/22

(57) ABSTRACT

The invention provides a human herpesvirus immunogenic peptide comprising a novel antigenic domain (AD) of glycoprotein B, termed AD-6. The invention also provides a nucleic acid sequence encoding said immunogenic peptide and an inhibitor that binds to said 5 immunogenic peptide. Also provided are an immunogenic composition, a pharmaceutical composition and a vaccine comprising said immunogenic peptide, nucleic acid sequence or inhibitor, and methods of treating or preventing a human herpesvirus infection.

### FIELD OF THE INVENTION

The invention relates to a human herpesvirus immunogenic peptide comprising a novel antigenic domain (AD) of glycoprotein B, termed AD-6. The invention also relates to a nucleic acid sequence encoding said immunogenic peptide and an inhibitor that binds to said immunogenic peptide. Also provided are an immunogenic composition, a pharmaceutical composition and a vaccine comprising said immunogenic peptide, nucleic acid sequence or inhibitor, and methods of treating or preventing a human herpesvirus infection.

### BACKGROUND TO THE INVENTION

Human herpesviruses can establish lifelong latent infections, and over 50 percent of the world's population are infected by such viruses. Reactivations of herpesvirus infection are usually controlled by a competent immune system; however, in persons with compromised or immature immune systems (e.g. immunosuppressed transplant patients, neonates or young children) herpesviruses can cause more serious disease and substantial morbidity is seen in multiple such patient populations. For example, the herpesvirus human cytomegalovirus (HCMV) is a leading cause of disease in congenital infection and immunosuppressed transplant patients and is also the most common viral cause of sensorineural hearing loss.

Treatment of infections caused by herpesviruses typically involves the use of systemic antiviral treatment, often administered orally or parenterally if there is a risk of disseminated or severe disease. For example, the main strategy for treating HCMV infections is the use of the nucleoside analogue antiviral drug ganciclovir (GCV) or its oral prodrug valganciclovir. Although GCV is effective at inhibiting HCMV DNA replication, it is associated with significant haematological and nephrological toxicity and there is increasing antiviral resistance to GCV. Alternative antivirals include Foscavir, which is given to individuals with GCV-resistant HCMV, but this also has notable nephrotoxicity. Another antiviral drug called letermovir has recently been licensed for prophylactic use immediately after stem cell transplant. Limitations of antiviral treatment alongside the financial and healthcare burden associated with HCMV infection has led the US Institute of Medicine to define HCMV as one of the 3 viral pathogens with the highest priority for vaccine and new therapeutic interventions.

One alternative treatment for herpesvirus infections is the use of immunoglobulin from seropositive individuals (cytomegalovirus immune globulin, CMVIG) to increase the immune response against HCMV. However, this treatment does not appear to work in all HCMV patient populations, for example paediatric lung transplant recipients.

Vaccines have been licensed for the prevention of certain herpesviruses, such as varicella zoster virus (VZV). However, there are no vaccines currently licensed for the prevention of other herpesviruses such as HCMV.

A candidate vaccine against HCMV that has recently been investigated is a composition of recombinant HCMV glycoprotein B (gB) with MF59 adjuvant. The gB protein elicits a strong humoral immune response in humans, although the majority of anti-gB antibodies produced are non-neutralising. Neutralising antibodies are the focus of conventional antibody-based vaccine research since they are expected to significantly reduce viral infectivity, whereas non-neutralising antibodies are typically not considered the most desirable response for protection against viral pathogens. In three patient populations (young mothers, Pass et al. 1999, J Infect Dis 180(4) 970-5; solid organ transplant recipients, Griffiths et al. 2011 Lancet 377 1256-63; and adolescent girls, Bernstein et al. 2016, Vaccine 34(3) 313-9) the gB with MF59 adjuvant candidate vaccine has been shown to be safe, immunogenic and generally well tolerated. However, with only approximately 40 to 50 percent efficacy or less the protection is not sufficient for licensing as an effective vaccine.

Thus, there is a need in the art for vaccines and treatments that induce an effective immune response against human herpesviruses.

### SUMMARY OF THE INVENTION

The present inventors have surprisingly identified a protective antibody response to a novel antigenic domain, termed antigenic domain (AD)-6, of HCMV gB. This antibody response is present in seronegative individuals vaccinated with recombinant HCMV gB protein but is rarely detected in seropositive (i.e. naturally infected) individuals. Surprisingly, the anti-AD-6 antibody response identified by the present inventors is capable of reducing the cell-to-cell spread of HCMV but is not a classical neutralising antibody. An immunogenic peptide that presents AD-6 to the immune system (e.g. in the context of an AD-6 subunit vaccine), particularly in the absence of other ADs of gB, has the potential to both focus the humoral response against AD-6 and prevent competing and potentially deleterious responses against other gB domains (e.g. other known ADs such as AD1-5). In other words, this approach is considered to be advantageous in promoting seronegative vaccine recipients to develop a robust antibody response against AD6. Furthermore, without wishing to be bound by theory, it is considered that such an immunogenic peptide will avoid the potential issue of original ‘antigenic sin’ that is seen with the candidate gB vaccine comprising recombinant HCMV gB and MF59 adjuvant in HCMV seropositive patients. This may be sufficient to improve the efficacy of a gB-based vaccine that can be utilised in both seronegative and seropositive individuals. Thus, the present invention is predicated upon the identification of a novel antigenic domain of gB that is considered to be effective for the prevention and treatment of herpesvirus infection.

In one aspect, the invention provides a human herpesvirus immunogenic peptide comprising SEQ ID NO: 7, SEQ ID NO: 9 or SEQ ID NO: 11, or a sequence having at least 75% (suitably, at least 80%, at least 85%, at least 90%, at least 95%, at least 96%, at least 97%, at least 98% or at least 99%) identity thereto, or an immunogenic fragment thereof.

In some embodiments, the human herpesvirus immunogenic peptide comprises SEQ ID NO: 7, a sequence having at least 75% (suitably, at least 80%, at least 85%, at least 90%, at least 95%, at least 96%, at least 97%, at least 98% or at least 99%) identity thereto, or an immunogenic fragment thereof.

In some embodiments, the human herpesvirus immunogenic peptide comprises SEQ ID NO: 9, a sequence having at least 75% (suitably, at least 80%, at least 85%, at least 90%, at least 95%, at least 96%, at least 97%, at least 98% or at least 99%) identity thereto, or an immunogenic fragment thereof.

In some embodiments, the human herpesvirus immunogenic peptide comprises SEQ ID NO: 11, a sequence having at least 75% (suitably, at least 80%, at least 85%, at least 90%, at least 95%, at least 96%, at least 97%, at least 98% or at least 99%) identity thereto, or an immunogenic fragment thereof.

In a further aspect, the invention provides an inhibitor of human herpesvirus glycoprotein-B which is capable of binding to human herpesvirus glycoprotein-B on the surface of a cell.

In some embodiments, said inhibitor binds within SEQ ID NO: 7, SEQ ID NO: 9 or SEQ ID NO: 11, or a sequence having at least 75% identity thereto, or a fragment thereof.

In a further aspect, the invention provides an inhibitor of human herpesvirus glycoprotein-B which is capable of binding to human herpesvirus glycoprotein-B, wherein the inhibitor binds within SEQ ID NO: 7, SEQ ID NO: 9 or SEQ ID NO: 11, or a sequence having at least 75% identity thereto, or a fragment thereof.

In some embodiments, said sequence has at least 80% identity to SEQ ID NO: 7, to SEQ ID NO: 9 or to SEQ ID NO: 11.

In some embodiments, said immunogenic fragment or said fragment is about 47 or fewer amino acids in length.

In some embodiments, said immunogenic fragment or said fragment is selected from SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25 or SEQ ID NO: 26, or a variant thereof having one, two or three amino acid substitutions and/or having one, two or three amino acid deletions at the N- or C-terminus.

In some embodiments, said immunogenic fragment or said fragment is SEQ ID NO: 25 or SEQ ID NO: 26 or a variant thereof having one, two or three amino acid substitutions and/or having one, two or three amino acid deletions at the N- or C-terminus.

In some embodiments, said human herpesvirus is human cytomegalovirus (HCMV), herpes simplex virus-1 (HSV-1), or Epstein-Barr virus (EBV)

In some embodiments, said human herpesvirus is HCMV.

In some embodiments, said inhibitor binds within any one of SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25 or SEQ ID NO: 26, or a fragment thereof, or a variant thereof having one, two or three amino acid substitutions and/or having one, two or three amino acid deletions at the N- and/or C-terminus.

In some embodiments, said inhibitor binds within SEQ ID NO: 25 or SEQ ID NO: 26, or a variant thereof having one, two or three amino acid substitutions and/or having one, two or three amino acid deletions at the N- or C-terminus.

In some embodiments, said inhibitor is selected from an antibody, an Ig fusion protein, a polypeptide, a peptide, a polynucleotide, a small molecule, a non-antibody scaffold, an aptamer, or combinations thereof.

In some embodiments, said inhibitor is an antibody.

In some embodiments, said antibody is a monoclonal antibody, a humanised antibody, a single-chain antibody, or an antibody fragment.

In a further aspect, the invention provides a nucleic acid sequence encoding the human herpesvirus immunogenic peptide of the invention.

In a further aspect, the invention provides a vector comprising the nucleic acid sequence of the invention.

In a further aspect, the invention provides an immunogenic composition comprising a human herpesvirus immunogenic peptide of the invention.

In a further aspect, the invention provides an immunogenic composition comprising a nucleic acid sequence of the invention or a vector of the invention.

In a further aspect, the invention provides a pharmaceutical composition comprising an inhibitor of the invention.

In a further aspect, the invention provides a vaccine comprising a human herpesvirus immunogenic peptide of the invention.

In a further aspect, the invention provides a vaccine comprising a nucleic acid sequence of the invention or a vector of the invention.

In a further aspect, the invention provides a vaccine comprising an inhibitor of the invention.

In a further aspect, the invention provides an immunogenic composition comprising a human herpesvirus immunogenic peptide of the invention for use in the treatment or prevention of human herpesvirus infection in a subject.

In a further aspect, the invention provides an immunogenic composition comprising a nucleic acid sequence of the invention or a vector of the invention for use in the treatment or prevention of human herpesvirus infection in a subject.

In a further aspect, the invention provides a pharmaceutical composition comprising an inhibitor of the invention for use in the treatment or prevention of human herpesvirus infection in a subject.

In a further aspect, the invention provides a vaccine comprising a human herpesvirus immunogenic peptide of the invention for use in the treatment or prevention of human herpesvirus infection in a subject.

In a further aspect, the invention provides a vaccine comprising a nucleic acid sequence of the invention or a vector of the invention for use in the treatment or prevention of human herpesvirus infection in a subject.

In a further aspect, the invention provides a vaccine comprising an inhibitor of the invention for use in the treatment or prevention of human herpesvirus infection in a subject.

In a further aspect, the invention provides an inhibitor of the invention for use in the treatment or prevention of human herpesvirus infection in a subject.

In some embodiments, the subject is:
a) an individual awaiting a solid organ transplant;
b) a solid organ transplant recipient;
c) a woman of childbearing age;
d) a pregnant woman; and/or
e) a child.

In some embodiments, the cell-to-cell spread of the human herpesvirus is inhibited.

In some embodiments, the immunogenic composition, pharmaceutical composition, vaccine or inhibitor is for use in the prevention of human herpesvirus infection in the subject.

In some embodiments, the human herpesvirus is human cytomegalovirus (HCMV), herpes simplex virus-1 (HSV-1), or Epstein-Barr virus (EBV)

In some embodiments, the human herpesvirus is HCMV.

In some embodiments, the immunogenic composition, pharmaceutical composition, vaccine or inhibitor is administered to the subject in combination with at least one further pharmaceutically active agent.

In some embodiments, the at least one further pharmaceutically active agent is selected from an antiviral, a herpesvirus vaccine, an immunostimulatory cytokine, a checkpoint inhibitor, TCR-gene-engineered T cells, activators of innate immunity, monoclonal or bispecific antibodies, CAR cells, soluble T cell receptors, or any combination thereof.

In some embodiments, the immunogenic composition, pharmaceutical composition, vaccine or inhibitor is administered simultaneously, sequentially or separately with the at least one further pharmaceutically active agent.

### BRIEF DESCRIPTION OF THE FIGURES

FIG. 1: Level of anti-gB antibody responses against linear peptides spanning aa648:697

FIG. 2: Diagram of gB protein structure

FIG. 3: Anti-AD6 antibody responses are rarely detected in naturally infected individuals

FIG. 4: Vaccinated individuals with an AD6 response have better outcomes post-transplant

FIG. 5: Generation of an anti-AD6 antibody in rabbits

FIG. 6: Anti-AD6 antibody does not neutralize CMV infection of fibroblasts

FIG. 7: Anti-AD6 antibody does not neutralize CMV infection of epithelial cells

FIG. 8: AD6 polyclonal sera (pAb) recognizes CMV infected cells

FIG. 9: Spread of HCMV is reduced in the presence of the AD6 antisera

FIG. 10: Anti-AD6 antisera limits cell associated spread of CMV. To measure spread of cell associated virus in HFFs, cells were infected with HCMV (MOI=0.01). At 1 dpi and 5 dpi, anti-AD6 antibody was added at the indicated concentrations and then 10 dpi, cells were then analysed by IF or DNA-qPCR for viral spread. Percentage of infected cells (A) was measured by anti-IE stain counterstained with nuclei stain and counted by automated fluorescence microscopy (n=3) and the area of plaques was measured using Fiji software (B). Alternatively, total DNA was harvested, and CMV genome copies per 10⁶ cells assessed by qPCR (C; n=2). ARPE-19 epithelial cells were infected with pentamer positive HCMV (MOI=0.01), and total DNA harvested 20dpi and analysed for viral genome copy number per 10⁶ cells (D). P values were calculated by a Kruskal-Wallis test with Dunn's multiple-comparison test where appropriate. ****, P<0.0001; ***,P<0.001; *, P<0.01; ns (nonsignificant), P>0.05. The error bars represent 1 standard deviation from the mean.

FIG. 11: A peptide within AD6 partially inhibits the activity of a polyclonal AD6 antisera against HCMV. HFFs were infected with HCMV (MOI=0.01). Then, at 1 d.p.i. and at 5 d.p.i, were incubated with anti-AD6 antibody (pAb), anti-AD6 antibody preabsorbed with AD6.4 peptide (pAb+AD6.4) or just peptide (AD6.4). The AD6.4 peptide spanned the N region of AD6 (gB₍₆₆₁₋₆₇₆₎) and was used at a 10× molar excess prior to addition to infected cells 1 d.p.i and at 5 d.p.i. Then, at 10 d.p.i. total DNA was harvested, and CMV genome copies per 10⁶ cells assessed by qPCR (n=2). The error bars represent standard error of the mean.

FIG. 12: Vaccine sera from patients with AD6 responses reduces viral spread and this is reversed by AD6 in some individuals. HFFs were infected with HCMV (MOI=0.01). Then, at 1 d.p.i. and at 5 d.p.i, we incubated with human sera from HCMV seronegative individuals who received placebo (A) or two doses of gB/MF59 vaccine (B,C). The capacity of sera and sera preabsorbed with AD6 peptide to limit viral spread was assessed at 10 d.p.i. when cells were analysed by IF to measure spread of the virus. Control represents spread of HCMV without the addition of sera.

### DETAILED DESCRIPTION OF THE INVENTION

### Immunogenic Peptide

The Herpesvirus family contains over 100 known viruses. There are nine herpesviruses that infect humans. Such human herpesviruses include herpes simplex virus-1 (HSV-1), Epstein-Barr virus (EBV) and human cytomegalovirus (HCMV).

Herpesviruses are double-stranded DNA enveloped viruses that can grow both cell-free and cell-associated. The viral life cycle involves the following major steps: viral attachment and entry into the host cell, viral gene expression, viral genome replication, virion assembly, and encapsidation and viral egress by exocytosis. Herpesviruses encode many viral glycoproteins, and many of these are critical for viral entry into the cell. Herpesvirus glycoprotein B (gB), a class III viral fusogen, is essential for viral entry into all host cell types and mediates the fusion of the viral envelope with the host cell membrane during entry and cell spread. The gB protein therefore has pre- and post-fusion conformations.

gB is the most highly conserved of all herpesvirus glycoproteins and occurs in all members of the herpesvirus family. The gB sequences of the human herpesviruses are known in the art.

An illustrative HCMV (strain AD169) gB amino acid sequence is provided by UniProtKB Accession Number P06473 and shown as SEQ ID NO: 1.

SEQ ID NO: 1
MESRIWCLVVCVNLCIVCLGAAVSSSSTSHATSSTHNGSHTSRTTSAQTR
SVYSQHVTSSEAVSHRANETIYNTTLKYGDVVGVNTTKYPYRVCSMAQGT
DLIRFERNIICTSMKPINEDLDEGIMVVYKRNIVAHTFKVRVYQKVLTFR
RSYAYIYTTYLLGSNTEYVAPPMWEIHHINKFAQCYSSYSRVIGGTVFVA
YHRDSYENKTMQLIPDDYSNTHSTRYVTVKDQWHSRGSTWLYRETCNLNC
MLTITTARSKYPYHFFATSTGDVVYISPFYNGTNRNASYFGENADKFFIF
PNYTIVSDFGRPNAAPETHRLVAFLERADSVISWDIQDEKNVTCQLTFWE
ASERTIRSEAEDSYHFSSAKMTATFLSKKQEVNMSDSALDCVRDEAINKL
QQIFNTSYNQTYEKYGNVSVFETSGGLVVFWQGIKQKSLVELERLANRSS
LNITHRTRRSTSDNNTTHLSSMESVHNLVYAQLQFTYDTLRGYINRALAQ
IAEAWCVDQRRTLEVFKELSKINPSAILSAIYNKPIAARFMGDVLGLASC
VTINQTSVKVLRDMNVKESPGRCYSRPVVIFNFANSSYVQYGQLGEDNEI
LLGNHRTEECQLPSLKIFIAGNSAYEYVDYLFKRMIDLSSISTVDSMIAL
DIDPLENTDFRVLELYSQKELRSSNVFDLEEIMREFNSYKQRVKYVEDKV
VDPLPPYLKGLDDLMSGLGAAGKAVGVAIGAVGGAVASVVEGVATFLKNP
FGAFTIILVAIAVVIITYLIYTRQRRLCTQPLQNLFPYLVSADGTTVTSG
STKDTSLQAPPSYEESVYNSGRKGPGPPSSDASTAAPPYTNEQAYQMLLA
LARLDAEQRAQQNGTDSLDGQTGTQDKGQKPNLLDRLRHRKNGYRHLKDS
DEEENV

An illustrative HCMV (strain AD169) gB polynucleotide sequence is provided by EMBL-EBI ENA Number AAA46009.1 and shown as SEQ ID NO: 2.

SEQ ID NO: 2
ATGGAATCCAGGATCTGGTGCCTGGTAGTCTGCGTTAACCTGTGTATCGT
CTGTCTGGGTGCTGCGGTTTCCTCTTCTAGTACTTCCCATGCAACTTCTT
CTACTCACAATGGAAGCCATACTTCTCGTACGACGTCTGCTCAAACCCGG
TCAGTCTATTCTCAACACGTAACGTCTTCTGAAGCCGTCAGTCATAGAGC
CAACGAGACTATCTACAACACTACCCTCAAGTACGGAGATGTGGTGGGAG
TCAACACTACCAAGTACCCCTATCGCGTGTGTTCTATGGCCCAGGGTACG
GATCTTATTCGCTTTGAACGTAATATCATCTGCACCTCGATGAAGCCTAT
CAATGAAGACTTGGATGAGGGCATCATGGTGGTCTACAAGCGCAACATCG
TGGCGCACACCTTTAAGGTACGGGTCTACCAAAAGGTTTTGACGTTTCGT
CGTAGCTACGCTTACATCTACACCACTTATCTGCTGGGCAGCAATACGGA
ATACGTGGCGCCTCCTATGTGGGAGATTCATCACATCAACAAGTTTGCTC
AATGCTACAGTTCCTACAGCCGCGTTATAGGAGGCACGGTTTTCGTGGCA
TATCATAGGGACAGTTATGAAAACAAAACCATGCAATTAATTCCCGACGA
TTATTCCAACACCCACAGTACCCGTTACGTGACGGTCAAGGATCAGTGGC
ACAGCCGCGGCAGCACCTGGCTCTATCGTGAGACCTGTAATCTGAACTGT
ATGCTGACCATCACTACTGCGCGCTCCAAGTATCCTTATCATTTTTTTGC
AACTTCCACGGGTGATGTGGTTTACATTTCTCCTTTCTACAACGGAACCA
ATCGCAATGCCAGCTACTTTGGAGAAAACGCCGACAAGTTTTTCATTTTC
CCGAACTACACCATCGTTTCCGACTTTGGAAGACCCAACGCTGCGCCAGA
AACCCATAGGTTGGTGGCTTTTCTCGAACGTGCCGACTCGGTGATCTCTT
GGGATATACAGGACGAGAAGAATGTCACCTGCCAGCTCACCTTCTGGGAA
GCCTCGGAACGTACTATCCGTTCCGAAGCCGAAGACTCGTACCACTTTTC
TTCTGCCAAAATGACTGCAACTTTTCTGTCTAAGAAACAAGAAGTGAACA
TGTCCGACTCCGCGCTGGACTGCGTACGTGATGAGGCTATAAATAAGTTA
CAGCAGATTTTCAATACTTCATACAATCAAACATATGAAAAATACGGAAA
CGTGTCCGTCTTCGAAACCAGCGGCGGTCTGGTGGTGTTCTGGCAAGGCA
TCAAGCAAAAATCTTTGGTGGAATTGGAACGTTTGGCCAATCGATCCAGT
CTGAATATCACTCATAGGACCAGAAGAAGTACGAGTGACAATAATACAAC
TCATTTGTCCAGCATGGAATCGGTGCACAATCTGGTCTACGCCCAGCTGC
AGTTCACCTATGACACGTTGCGCGGTTACATCAACCGGGCGCTGGCGCAA
ATCGCAGAAGCCTGGTGTGTGGATCAACGGCGCACCCTAGAGGTCTTCAA
GGAACTCAGCAAGATCAACCCGTCAGCCATTCTCTCGGCCATTTACAACA
AACCGATTGCCGCGCGTTTCATGGGTGATGTCTTGGGCCTGGCCAGCTGC
GTGACCATCAACCAAACCAGCGTCAAGGTGCTGCGTGATATGAACGTGAA
GGAATCGCCAGGACGCTGCTACTCACGACCCGTGGTCATCTTTAATTTCG
CCAACAGCTCGTACGTGCAGTACGGTCAACTGGGCGAGGACAACGAAATC
CTGTTGGGCAACCACCGCACTGAGGAATGTCAGCTTCCCAGCCTCAAGAT
CTTCATCGCCGGGAACTCGGCCTACGAGTACGTGGACTACCTCTTCAAAC
GCATGATTGACCTCAGCAGTATCTCCACCGTCGACAGCATGATCGCCCTG
GATATCGACCCGCTGGAAAATACCGACTTCAGGGTACTGGAACTTTACTC
GCAGAAAGAGCTGCGTTCCAGCAACGTTTTTGACCTCGAAGAGATCATGC
GCGAATTCAACTCGTACAAGCAGCGGGTAAAGTACGTGGAGGACAAGGTA
GTCGACCCGCTACCGCCCTACCTCAAGGGTCTGGACGACCTCATGAGCGG
CCTGGGCGCCGCGGGAAAGGCCGTTGGCGTAGCCATTGGGGCCGTGGGTG
GCGCGGTGGCCTCCGTGGTCGAAGGCGTTGCCACCTTCCTCAAAAACCCC
TTCGGAGCCTTCACCATCATCCTCGTGGCCATAGCCGTAGTCATTATCAC
TTATTTGATCTATACTCGACAGCGGCGTCTGTGCACGCAGCCGCTGCAGA
ACCTCTTTCCCTATCTGGTGTCCGCCGACGGGACCACCGTGACGTCGGGC
AGCACCAAAGACACGTCGTTACAGGCTCCGCCTTCCTACGAGGAAAGTGT
TTATAATTCTGGTCGCAAAGGACCGGGACCACCGTCGTCTGATGCATCCA
CGGCGGCTCCGCCTTACACCAACGAGCAGGCTTACCAGATGCTTCTGGCC
CTGGCCCGTCTGGACGCAGAGCAGCGAGCGCAGCAGAACGGTACAGATTC
TTTGGACGGACAGACTGGCACGCAGGACAAGGGACAGAAGCCTAACCTGC
TAGACCGGCTGCGACATCGCAAAAACGGCTACAGACACTTGAAAGACTCC
GACGAAGAAGAGAACGTCTGA

A further illustrative HCMV (strain Merlin) gB amino acid sequence is provided by UniProtKB Accession Number F5HB53 and shown as SEQ ID NO: 27.

SEQ ID NO: 27
MESRIWCLVVCVNLCIVCLGAAVSSSSTRGTSATHSHHSSHTTSAAHSRS
GSVSQRVTSSQTVSHGVNETIYNTTLKYGDVVGVNTTKYPYRVCSMAQGT
DLIRFERNIVCTSMKPINEDLDEGIMVVYKRNIVAHTFKVRVYQKVLTFR
RSYAYIHTTYLLGSNTEYVAPPMWEIHHINSHSQCYSSYSRVIAGTVFVA
YHRDSYENKTMQLMPDDYSNTHSTRYVTVKDQWHSRGSTWLYRETCNLNC
MVTITTARSKYPYHFFATSTGDVVDISPFYNGTNRNASYFGENADKFFIF
PNYTIVSDFGRPNSALETHRLVAFLERADSVISWDIQDEKNVTCQLTFWE
ASERTIRSEAEDSYHFSSAKMTATFLSKKQEVNMSDSALDCVRDEAINKL
QQIFNTSYNQTYEKYGNVSVFETTGGLVVFWQGIKQKSLVELERLANRSS
LNLTHNRTKRSTDGNNATHLSNMESVHNLVYAQLQFTYDTLRGYINRALA
QIAEAWCVDQRRTLEVFKELSKINPSAILSAIYNKPIAARFMGDVLGLAS
CVTINQTSVKVLRDMNVKESPGRCYSRPVVIFNFANSSYVQYGQLGEDNE
ILLGNHRTEECQLPSLKIFIAGNSAYEYVDYLFKRMIDLSSISTVDSMIA
LDIDPLENTDFRVLELYSQKELRSSNVFDLEEIMREFNSYKQRVKYVEDK
VVDPLPPYLKGLDDLMSGLGAAGKAVGVAIGAVGGAVASVVEGVATFLKN
PFGAFTIILVAIAVVIITYLIYTRORRLCTQPLQNLFPYLVSADGTTVTS
GSTKDTSLQAPPSYEESVYNSGRKGPGPPSSDASTAAPPYTNEQAYQMLL
ALARLDAEQRAQQNGTDSLDGRTGTQDKGQKPNLLDRLRHRKNGYRHLKD
SDEEENV

A further illustrative HCMV (strain Merlin) gB polynucleotide sequence is provided by EMBL-EBI ENA Number AAR31620.1 and shown as SEQ ID NO: 28.

SEQ ID NO: 28
ATGGAATCCAGGATCTGGTGCCTGGTAGTCTGCGTTAACTTGTGTATCGT
CTGTCTGGGTGCTGCGGTTTCCTCATCTTCTACTCGTGGAACTTCTGCTA
CTCACAGTCACCATTCCTCTCATACGACGTCTGCTGCTCACTCTCGATCC
GGTTCAGTCTCTCAACGCGTAACTTCTTCCCAAACGGTCAGCCATGGTGT
TAACGAGACCATCTACAACACTACCCTCAAGTACGGAGATGTGGTGGGGG
TCAATACCACCAAGTACCCCTATCGCGTGTGTTCTATGGCCCAGGGTACG
GATCTTATTCGCTTTGAACGTAATATCGTCTGCACCTCGATGAAGCCCAT
CAATGAAGACCTGGACGAGGGCATCATGGTGGTCTACAAACGCAACATCG
TCGCGCACACCTTTAAGGTACGAGTCTACCAGAAGGTTTTGACGTTTCGT
CGTAGCTACGCTTACATCCACACCACTTATCTGCTGGGCAGCAACACGGA
ATACGTGGCGCCTCCTATGTGGGAGATTCATCATATCAACAGCCACAGTC
AGTGCTACAGTTCCTACAGCCGCGTTATAGCAGGCACGGTTTTCGTGGCT
TATCATAGGGACAGCTATGAAAACAAAACCATGCAATTAATGCCCGACGA
TTATTCCAACACCCACAGTACCCGTTACGTGACGGTCAAGGATCAATGGC
ACAGCCGCGGCAGCACCTGGCTCTATCGTGAGACCTGTAATCTGAATTGT
ATGGTGACCATCACTACTGCGCGCTCCAAATATCCTTATCATTTTTTCGC
CACTTCCACGGGTGACGTGGTTGACATTTCTCCTTTCTACAACGGAACCA
ATCGCAATGCCAGCTACTTTGGAGAAAACGCCGACAAGTTTTTCATTTTT
CCGAACTACACTATCGTCTCCGACTTTGGAAGACCGAATTCTGCGTTAGA
GACCCACAGGTTGGTGGCTTTTCTTGAACGTGCGGACTCGGTGATCTCCT
GGGATATACAGGACGAAAAGAATGTCACTTGTCAACTCACTTTCTGGGAA
GCCTCGGAACGCACCATTCGTTCCGAAGCCGAGGACTCGTATCACTTTTC
TTCTGCCAAAATGACCGCCACTTTCTTATCTAAGAAGCAAGAGGTGAACA
TGTCCGACTCTGCGCTGGACTGCGTACGTGATGAGGCTATAAATAAGTTA
CAGCAGATTTTCAATACTTCATACAATCAAACATATGAAAAATATGGAAA
CGTGTCCGTCTTTGAAACCACTGGTGGTTTGGTAGTGTTCTGGCAAGGTA
TCAAGCAAAAATCTCTGGTGGAACTCGAACGTTTGGCCAACCGCTCCAGT
CTGAATCTTACTCATAATAGAACCAAAAGAAGTACAGATGGCAACAATGC
AACTCATTTATCCAACATGGAATCGGTGCACAATCTGGTCTACGCCCAGC
TGCAGTTCACCTATGACACGTTGCGCGGTTACATCAACCGGGCGCTGGCG
CAAATCGCAGAAGCCTGGTGTGTGGATCAACGGCGCACCCTAGAGGTCTT
CAAGGAACTCAGCAAGATCAACCCGTCAGCCATTCTCTCGGCCATTTACA
ACAAACCGATTGCCGCGCGTTTCATGGGTGATGTCTTGGGCCTGGCCAGC
TGCGTGACCATCAACCAAACCAGCGTCAAGGTGCTGCGTGATATGAACGT
GAAGGAGTCGCCAGGACGCTGCTACTCACGACCCGTGGTCATCTTTAATT
TCGCCAACAGCTCGTACGTGCAGTACGGTCAACTGGGCGAGGACAACGAA
ATCCTGTTGGGCAACCACCGCACTGAGGAATGTCAGCTTCCCAGCCTCAA
GATCTTCATCGCCGGGAACTCGGCCTACGAGTACGTGGACTACCTCTTCA
AACGCATGATTGACCTCAGCAGTATCTCCACCGTCGACAGCATGATCGCC
CTGGATATCGACCCGCTGGAAAATACCGACTTCAGGGTACTGGAACTTTA
CTCGCAGAAAGAGCTGCGTTCCAGCAACGTTTTTGACCTCGAAGAGATCA
TGCGCGAATTCAACTCGTACAAGCAGCGGGTAAAGTACGTGGAGGACAAG
GTAGTCGACCCGCTACCGCCCTACCTCAAGGGTCTGGACGACCTCATGAG
CGGCCTGGGCGCCGCGGGAAAGGCCGTTGGCGTAGCCATTGGGGCCGTGG
GTGGCGCGGTGGCCTCCGTGGTCGAAGGCGTTGCCACCTTCCTCAAAAAC
CCCTTCGGAGCGTTCACCATCATCCTCGTGGCCATAGCTGTAGTCATTAT
CACTTATTTGATCTATACTCGACAGCGGCGTTTGTGCACGCAGCCGCTGC
AGAACCTCTTTCCCTATCTGGTGTCCGCCGACGGGACCACCGTGACGTCG
GGCAGCACCAAAGACACGTCGTTACAGGCTCCGCCTTCCTACGAGGAAAG
TGTTTATAATTCTGGTCGCAAAGGACCGGGACCACCGTCGTCTGATGCAT
CCACGGCGGCTCCGCCTTACACCAACGAGCAGGCTTACCAGATGCTTCTG
GCCCTGGCCCGTCTGGACGCAGAGCAGCGAGCGCAGCAGAACGGTACAGA
TTCTTTGGACGGACGGACTGGCACGCAGGACAAGGGACAGAAGCCCAACC
TACTAGACCGACTGCGACATCGCAAAAACGGCTACCGACACTTGAAAGAC
TCTGACGAAGAAGAGAACGTCTGA

An illustrative HSV-1 (strain KOS) gB amino acid sequence is provided by UniProtKB Accession Number P06437 and shown as SEQ ID NO: 3.

SEQ ID NO: 3
MHQGAPSWGRRWFVVWALLGLTLGVLVASAAPTSPGTPGVAAATQAANGG
PATPAPPPLGAAPTGDPKPKKNKKPKNPTPPRPAGDNATVAAGHATLREH
LRDIKAENTDANFYVCPPPTGATVVQFEQPRRCPTRPEGQNYTEGIAVVF
KENIAPYKFKATMYYKDVTVSQVWFGHRYSQFMGIFEDRAPVPFEEVIDK
INAKGVCRSTAKYVRNNLETTAFHRDDHETDMELKPANAATRTSRGWHTT
DLKYNPSRVEAFHRYGTTVNCIVEEVDARSVYPYDEFVLATGDFVYMSPF
YGYREGSHTEHTTYAADRFKQVDGFYARDLTTKARATAPTTRNLLTTPKF
TVAWDWVPKRPSVCTMTKWQEVDEMLRSEYGGSFRFSSDAISTTFTTNLT
EYPLSRVDLGDCIGKDARDAMDRIFARRYNATHIKVGQPQYYQANGGFLI
AYQPLLSNTLAELYVREHLREQSRKPPNPTPPPPGASANASVERIKTTSS
IEFARLQFTYNHIQRHVNDMLGRVAIAWCELQNHELTLWNEARKLNPNAI
ASVTVGRRVSARMLGDVMAVSTCVPVAADNVIVQNSMRISSRPGACYSRP
LVSFRYEDQGPLVEGQLGENNELRLTRDAIEPCTVGHRRYFTFGGGYVYF
EEYAYSHQLSRADITTVSTFIDLNITMLEDHEFVPLEVYTRHEIKDSGLL
DYTEVQRRNQLHDLRFADIDTVIHADANAAMFAGLGAFFEGMGDLGRAVG
KVVMGIVGGVVSAVSGVSSFMSNPFGALAVGLLVLAGLAAAFFAFRYVMR
LQSNPMKALYPLTTKELKNPTNPDASGEGEEGGDFDEAKLAEAREMIRYM
ALVSAMERTEHKAKKKGTSALLSAKVTDMVMRKRRNTNYTQVPNKDGDAD
EDDL

An illustrative HSV-1 (strain KOS) gB polynucleotide sequence is provided by EMBL-EBI ENA Number AAA45774.1 and shown as SEQ ID NO: 4.

SEQ ID NO: 4
ATGCACCAGGGCGCCCCCTCGTGGGGGCGCCGGTGGTTCGTCGTATGGGC
GCTCTTGGGGTTGACGCTGGGGGTCCTGGTGGCGTCGGCGGCTCCGACTT
CCCCCGGCACGCCTGGGGTCGCGGCCGCGACCCAGGCGGCGAACGGGGGC
CCTGCCACTCCGGCGCCGCCGCCCCTTGGCGCCGCCCCAACGGGGGACCC
GAAACCGAAGAAGAACAAAAAACCGAAAAACCCAACGCCACCACGCCCCG
CCGGCGACAACGCGACCGTCGCCGCGGGCCACGCCACCCTGCGCGAGCAC
CTGCGGGACATCAAGGCGGAGAACACCGATGCAAACTTTTACGTGTGCCC
ACCCCCCACGGGCGCCACGGTGGTGCAGTTCGAGCAGCCGCGCCGCTGCC
CGACCCGGCCCGAGGGTCAGAACTACACGGAGGGCATCGCGGTGGTCTTC
AAGGAGAACATCGCCCCGTACAAGTTCAAGGCCACCATGTACTACAAAGA
CGTCACCGTTTCGCAGGTGTGGTTCGGCCACCGCTACTCCCAGTTTATGG
GGATCTTTGAGGACCGCGCCCCCGTCCCCTTCGAGGAGGTGATCGACAAG
ATCAACGCCAAGGGGGTCTGTCGGTCCACGGCCAAGTACGTGCGCAACAA
CCTGGAGACCACCGCGTTTCACCGGGACGACCACGAGACCGACATGGAGC
TGAAACCGGCCAACGCCGCGACCCGCACGAGCCGGGGCTGGCACACCACC
GACCTCAAGTACAACCCCTCGCGGGTGGAGGCGTTCCACCGGTACGGGAC
GACGGTAAACTGCATCGTCGAGGAGGTGGACGCGCGCTCGGTGTACCCGT
ACGACGAGTTTGTGCTGGCGACTGGCGACTTTGTGTACATGTCCCCGTTT
TACGGCTACCGGGAGGGGTCGCACACCGAACACACCACGTACGCCGCCGA
CCGCTTCAAGCAGGTCGACGGCTTCTACGCGCGCGACCTCACCACCAAGG
CCCGGGCCACGGCGCCGACCACCCGGAACCTGCTCACGACCCCCAAGTTC
ACCGTGGCCTGGGACTGGGTGCCAAAGCGCCCGTCGGTCTGCACCATGAC
CAAGTGGCAGGAAGTGGACGAGATGCTGCGCTCCGAGTACGGCGGCTCCT
TCCGATTCTCCTCCGACGCCATATCCACCACCTTCACCACCAACCTGACC
GAGTACCCGCTCTCGCGCGTGGACCTGGGGGACTGCATCGGCAAGGACGC
CCGCGACGCCATGGACCGCATCTTCGCCCGCAGGTACAACGCGACGCACA
TCAAGGTGGGCCAGCCGCAGTACTACCAGGCCAATGGGGGCTTTCTGATC
GCGTACCAGCCCCTTCTCAGCAACACGCTCGCGGAGCTGTACGTGCGGGA
ACACCTCCGAGAGCAGAGCCGCAAGCCCCCAAACCCCACGCCCCCGCCGC
CCGGGGCCAGCGCCAACGCGTCCGTGGAGCGCATCAAGACCACCTCCTCC
ATCGAGTTCGCCCGGCTGCAGTTTACGTACAACCACATACAGCGCCATGT
CAACGATATGTTGGGCCGCGTTGCCATCGCGTGGTGCGAGCTACAGAATC
ACGAGCTGACCCTGTGGAACGAGGCCCGCAAGCTGAACCCCAACGCCATC
GCCTCGGTCACCGTGGGCCGGCGGGTGAGCGCGCGGATGCTCGGCGACGT
GATGGCCGTCTCCACGTGCGTGCCGGTCGCCGCGGACAACGTGATCGTCC
AAAACTCGATGCGCATCAGCTCGCGGCCCGGGGCCTGCTACAGCCGCCCC
CTGGTCAGCTTTCGGTACGAAGACCAGGGCCCGTTGGTCGAGGGGCAGCT
GGGGGAGAACAACGAGCTGCGGCTGACGCGCGATGCGATCGAGCCGTGCA
CCGTGGGACACCGGCGCTACTTCACCTTCGGTGGGGGCTACGTGTACTTC
GAGGAGTACGCGTACTCCCACCAGCTGAGCCGCGCCGACATCACCACCGT
CAGCACCTTCATCGACCTCAACATCACCATGCTGGAGGATCACGAGTTTG
TCCCCCTGGAGGTGTACACCCGCCACGAGATCAAGGACAGCGGCCTGCTG
GACTACACGGAGGTCCAGCGCCGCAACCAGCTGCACGACCTGCGCTTCGC
CGACATCGACACGGTCATCCACGCCGACGCCAACGCCGCCATGTTCGCGG
GCCTGGGCGCGTTCTTCGAGGGGATGGGCGACCTGGGGCGCGCGGTCGGC
AAGGTGGTGATGGGCATCGTGGGCGGCGTGGTATCGGCCGTGTCGGGCGT
GTCCTCCTTCATGTCCAACCCCTTTGGGGCGCTGGCCGTGGGTCTGTTGG
TCCTGGCCGGCCTGGCGGCGGCCTTCTTCGCCTTTCGTTACGTCATGCGG
CTGCAGAGCAACCCCATGAAGGCCCTGTACCCTCTAACCACCAAGGAGCT
CAAGAACCCCACCAACCCGGACGCGTCCGGGGAGGGCGAGGAGGGCGGCG
ACTTTGACGAGGCCAAGCTAGCCGAGGCCAGGGAGATGATACGGTACATG
GCCCTGGTGTCGGCCATGGAGCGCACGGAACACAAGGCCAAGAAGAAGGG
CACGAGCGCGCTGCTCAGCGCCAAGGTCACCGACATGGTCATGCGCAAGC
GCCGCAACACCAACTACACCCAAGTTCCCAACAAAGACGGTGACGCCGAC
GAGGACGACCTGTGA

An illustrative EBV (strain B958) gB amino acid sequence is provided by Uniprot Accession Number Q777130 and shown as SEQ ID NO: 5.

SEQ ID NO: 5
MTRRRVLSVVVLLAALACRLGAQTPEQPAPPATTVQPTATRQQTSFPFRV
CELSSHGDLFRFSSDIQCPSFGTRENHTEGLLMVFKDNIIPYSFKVRSYT
KIVTNILIYNGWYADSVTNRHEEKFSVDSYETDQMDTIYQCYNAVKMTKD
GLTRVYVDRDGVNITVNLKPTGGLANGVRRYASQTELYDAPGWLIWTYRT
RTTVNCLITDMMAKSNSPFDFFVTTTGQTVEMSPFYDGKNKETFHERADS
FHVRTNYKIVDYDNRGTNPQGERRAFLDKGTYTLSWKLENRTAYCPLQHW
QTFDSTIATETGKSIHFVTDEGTSSFVTNTTVGIELPDAFKCIEEQVNKT
MHEKYEAVQDRYTKGQEAITYFITSGGLLLAWLPLTPRSLATVKNLTELT
TPTSSPPSSPSPPAPSAARGSTPAAVLRRRRRDAGNATTPVPPTAPGKSL
GTLNNPATVQIQFAYDSLRRQINRMLGDLARAWCLEQKRQNMVLRELTKI
NPTTVMSSIYGKAVAAKRLGDVISVSQCVPVNQATVTLRKSMRVPGSETM
CYSRPLVSFSFINDTKTYEGQLGTDNEIFLTKKMTEVCQATSQYYFQSGN
EIHVYNDYHHFKTIELDGIATLQTFISLNTSLIENIDFASLELYSRDEQR
ASNVFDLEGIFREYNFQAQNIAGLRKDLDNAVSNGRNQFVDGLGELMDSL
GSVGQSITNLVSTVGGLFSSLVSGFISFFKNPFGGMLILVLVAGVVILVI
SLTRRTRQMSQQPVQMLYPGIDELAQQHASGEGPGINPISKTELQAIMLA
LHEQNQEQKRAAQRAAGPSVASRALQAARDRFPGLRRRRYHDPETAAALL
GEAETEF

An illustrative EBV (strain B958) gB polynucleotide sequence is provided by EMBL-EBI ENA Number CAA24806.1 and shown as SEQ ID NO: 6.

SEQ ID NO: 6
ATGACTCGGCGTAGGGTGCTAAGCGTGGTCGTGCTGCTAGCCGCCCTGGC
GTGCCGTCTCGGTGCGCAGACCCCAGAGCAGCCCGCACCCCCCGCCACCA
CGGTGCAGCCTACCGCCACGCGTCAGCAAACCAGCTTTCCTTTCCGAGTC
TGCGAGCTCTCCAGCCACGGCGACCTGTTCCGCTTCTCCTCGGACATCCA
GTGTCCCTCGTTTGGCACGCGGGAGAATCACACGGAGGGCCTGTTGATGG
TGTTTAAAGACAACATTATTCCCTACTCGTTTAAGGTCCGCTCCTACACC
AAGATAGTGACCAACATTCTCATCTACAATGGCTGGTACGCGGACTCCGT
GACCAACCGGCACGAGGAGAAGTTCTCCGTTGACAGCTACGAAACTGACC
AGATGGATACCATCTACCAGTGCTACAACGCGGTCAAGATGACAAAAGAT
GGGCTGACGCGCGTGTATGTAGACCGCGACGGAGTTAACATCACCGTCAA
CCTAAAGCCCACCGGGGGCCTGGCCAACGGGGTGCGCCGCTACGCCAGCC
AGACGGAGCTCTATGACGCCCCCGGGTGGTTGATATGGACTTACAGAACA
AGAACTACCGTCAACTGCCTGATAACTGACATGATGGCCAAGTCCAACAG
CCCCTTCGACTTCTTTGTGACCACCACCGGGCAGACTGTGGAAATGTCCC
CTTTCTATGACGGGAAAAATAAGGAAACCTTCCATGAGCGGGCAGACTCC
TTCCACGTGAGAACTAACTACAAGATAGTGGACTACGACAACCGAGGGAC
GAACCCGCAAGGCGAACGCCGAGCCTTCCTGGACAAGGGCACTTACACGC
TATCTTGGAAGCTCGAGAACAGGACAGCCTACTGCCCGCTTCAACACTGG
CAAACCTTTGACTCGACCATCGCCACAGAAACAGGGAAGTCAATACATTT
TGTGACTGACGAGGGCACCTCTAGCTTCGTGACCAACACAACCGTGGGCA
TAGAGCTCCCGGACGCCTTCAAGTGCATCGAAGAGCAGGTGAACAAGACC
ATGCATGAGAAGTACGAGGCCGTCCAGGATCGTTACACGAAGGGCCAGGA
AGCCATTACATATTTTATAACGAGCGGAGGATTGTTATTAGCTTGGCTAC
CTCTGACCCCGCGCTCGTTGGCCACCGTCAAGAACCTGACGGAGCTTACC
ACTCCGACTTCCTCACCCCCCAGCAGTCCATCGCCCCCAGCCCCATCCGC
GGCCCGCGGGAGCACCCCCGCCGCCGTTCTGAGGCGTCGGAGGCGGGATG
CGGGGAACGCCACCACACCGGTGCCCCCCACGGCCCCCGGGAAGTCCCTG
GGCACCCTCAACAATCCCGCCACCGTCCAGATCCAATTTGCCTACGACTC
CCTGCGCCGCCAGATCAACCGCATGCTGGGAGACCTTGCGCGGGCCTGGT
GCCTGGAGCAGAAGAGGCAGAACATGGTGCTGAGAGAACTAACCAAGATT
AATCCAACCACCGTCATGTCCAGCATCTACGGTAAGGCGGTGGCGGCCAA
GCGCCTGGGGGATGTCATCTCAGTCTCCCAGTGCGTGCCCGTTAACCAGG
CCACCGTCACCCTGCGCAAGAGCATGAGGGTCCCTGGCTCCGAGACCATG
TGCTACTCGCGCCCCCTGGTGTCCTTCAGCTTTATCAACGACACCAAGAC
CTACGAGGGACAGCTGGGCACCGACAACGAGATCTTCCTCACAAAAAAGA
TGACGGAGGTGTGCCAGGCGACCAGCCAGTACTACTTCCAGTCCGGCAAC
GAGATCCACGTCTACAACGACTACCACCACTTTAAAACCATCGAGCTGGA
CGGCATTGCCACCCTGCAGACCTTCATCTCACTAAACACCTCCCTCATCG
AGAACATTGACTTTGCCTCCCTGGAGCTGTACTCACGGGACGAACAGCGT
GCCTCCAACGTCTTTGACCTGGAGGGCATCTTCCGGGAGTACAACTTCCA
GGCGCAAAACATCGCCGGCCTGCGGAAGGATTTGGACAATGCAGTGTCAA
ACGGAAGAAATCAATTCGTGGACGGCCTGGGGGAACTTATGGACAGTCTG
GGTAGCGTGGGTCAGTCCATCACCAACCTAGTCAGCACGGTGGGGGGTTT
GTTTAGCAGCCTGGTCTCTGGTTTCATCTCCTTCTTCAAAAACCCCTTCG
GCGGCATGCTCATTCTGGTCCTGGTGGCGGGGGTGGTGATCCTGGTTATT
TCCCTCACGAGGCGCACGCGCCAGATGTCGCAGCAGCCGGTGCAGATGCT
CTACCCCGGGATCGACGAGCTCGCTCAGCAACATGCCTCTGGTGAGGGTC
CAGGCATTAATCCCATTAGTAAGACAGAATTACAAGCCATCATGTTAGCG
CTGCATGAGCAAAACCAGGAGCAAAAGAGAGCAGCTCAGAGGGCGGCCGG
ACCCTCAGTGGCCAGCAGAGCATTGCAGGCAGCCAGGGACCGTTTTCCAG
GCCTACGCAGAAGACGCTATCACGATCCAGAGACCGCCGCCGCACTGCTT
GGGGAGGCAGAGACTGAGTTTTA

gB is a large, glycosylated protein of approximately 900 amino acids. The crystal structure of the gB ectodomain from several herpesviruses (including HSV-1, EBV and HCMV) has been solved as a trimer in its post-fusion conformation, revealing five structural domains (Domains I-V) (Heldwein EE et al. (2006), Science, 313:217-220; Backovic M et al. (2009), Proc. Natl. Acad. Sci. U.S.A, 106:2880-2885; and Burke, HG and Heldwein, EE (2015), PLoS Pathogens, 11(10):e1005300). Domain III (also known as the core domain) is a long helix that forms a central triple coiled coil within gB trimer, whilst Domain V spans almost the full length of the gB ectodomain and fits into a groove formed by Domain III and Domain I of the other two gB monomers within the trimer structure. The pre-fusion conformation of a herpesvirus gB has not yet been characterised by crystallography. Despite the relatively low sequence identity between the various human herpesvirus gBs, the crystal structures of the gB ectodomains are very similar.

The major antigenic domains (AD) of HSV-1 and HCMV gB during natural infection have been identified, and in both cases multiple competing ADs exist. The five major ADs identified in HCMV gB protein are: AD-1, AD-2, AD-3, AD-4 and AD-5. Of these, AD-2, AD-4 and AD-5, have been considered the most promising candidates for production of therapeutic immunoglobulins (Burke, HG and Heldwein, EE (2015), PLoS Pathogens, 11(10):e1005300). None of AD-1 to AD-5 are located within Domain III or Domain V of HCMV gB, and no antibodies against Domains III or V of HCMV gB have been previously isolated.

As used herein, the term “antigenic domain (AD)” means a target sequence or structure within an antigen, e.g. within gB, that is recognised by antibodies. Thus, an antigenic domain contains a limited number of neighbouring epitopes recognised by antibodies, for example, in infected or vaccinated individuals.

gB is the dominant antigenic protein within the herpesvirus envelope and elicits a strong humoral immune response in humans, although the majority of anti-gB antibodies are non-neutralising and target the immunodominant antigenic site AD-1. Neutralising antibodies are the focus of conventional antibody-based vaccine research since they are expected to significantly reduce viral infectivity, whereas non-neutralising antibodies are typically not thought to offer significant protection against viral pathogens. The lack of neutralising antibodies to gB has been attributed to the diversion of the immune response towards production of non-neutralising antibodies at multiple competing antigenic sites by herpesviruses. This can be achieved, for example, by glycan shielding of certain epitopes to protect them from the immune system—whilst HCMV gB is heavily glycosylated, there is limited glycosylation of Domain IV which contains the immunodominant site AD-1.

The present inventors have surprisingly identified a novel AD of gB, referred to as “AD-6” or “AD6” herein. This novel AD is recognised by antibodies in vaccinated seronegative individuals, but only rarely in vaccinated seropositive (i.e. naturally infected) individuals. In some embodiments, AD-6 maps to amino acids 647 to 694 of SEQ ID NO: 1 or to amino acids 648 to 695 of SEQ ID NO: 27, and so spans two structural domains, Domain III and Domain V, of HCMV gB.

An immunogenic peptide of the invention that presents AD-6 to the immune system (e.g. in the context of an AD-6 subunit vaccine) has the potential to be effective, particularly if the presentation of other ADs of gB is absent. This would allow the immune system to focus on generating humoral responses against the novel AD-6 epitope rather than potential “decoy” epitopes (e.g. AD-1, AD-2, AD-3, AD-4 and/or AD-5) present in gB, i.e. to avoid the issue of original ‘antigenic sin’ that is seen in HCMV seropositive patients with the candidate gB vaccine comprising recombinant HCMV gB and MF59 adjuvant (Baraniak et al. 2019 J. Infect. Dis. 220(2) 228-232). Thus, an immunogenic peptide that presents AD-6 to the immune system (e.g. in the context of an AD-6 subunit vaccine) would both focus the humoral response against AD-6 and prevent deleterious response against other gB domains. This may be sufficient to improve the efficacy of a gB-based vaccine.

The identification of a virus having a protein with an AD-6 domain can be performed using methods known in the art. For example, a suitable virus can be identified by the ability of an anti-AD-6 antibody to prevent virus infection and replication in vitro, for example using an assay as described herein (see Example 2). The assay may be performed in any cell type that supports lytic HCMV replication (e.g. HFF, ARPE, U373, dendritic cell, macrophage, endothelial cell). It is within the capabilities of the skilled person to select a suitable cell type.

The identification of an AD-6 domain can be performed using methods known in the art. Suitably, the ability of an anti-AD-6 antibody to bind the candidate domain (e.g. a candidate gB protein) may be determined using an isolated candidate domain (e.g. recombinant protein). Suitable methods for determining binding will be known to those of skill in the art. For example, binding may be assessed by ELISA, flow cytometry, immunohistochemistry, Western blotting and surface plasmon resonance. Preferably, binding may be assessed by ELISA. An anti-AD-6 antibody may be generated using methods known in the art, for example as described herein (see Example 2). It is within the ambit of the skilled person to select and implement a suitable assay to determine if a candidate domain is an AD-6 domain.

An illustrative HCMV gB AD-6 amino acid sequence is shown as SEQ ID NO: 7.

SEQ ID NO: 7
MIALDIDPLENTDFRVLELYSQKELRSSNVFDLEEIMREFNSYKQRVK

An illustrative HCMV gB AD-6 nucleic acid sequence is shown as SEQ ID NO: 8.

SEQ ID NO: 8
ATGATCGCCCTGGATATCGACCCGCTGGAAAATACCGACTTCAGGGTACT
GGAACTTTACTCGCAGAAAGAGCTGCGTTCCAGCAACGTTTTTGACCTCG
AAGAGATCATGCGCGAATTCAACTCGTACAAGCAGCGGGTAAAG

An illustrative HSV-1 gB AD-6 amino acid sequence is shown as SEQ ID NO: 9.

SEQ ID NO: 9
FIDLNITMLEDHEFVPLEVYTRHEIKDSGLLDYTEVQRRNQLHDLRFA

An illustrative HSV-1 gB AD-6 nucleic acid sequence is shown as SEQ ID NO: 10.

SEQ ID NO: 10
TTCATCGACCTCAACATCACCATGCTGGAGGATCACGAGTTTGTCCCCCT
GGAGGTGTACACCCGCCACGAGATCAAGGACAGCGGCCTGCTGGACTACA
CGGAGGTCCAGCGCCGCAACCAGCTGCACGACCTGCGCTTCGCC

An illustrative EBV gB AD-6 amino acid sequence is shown as SEQ ID NO: 11.

SEQ ID NO: 11
FISLNTSLIENIDFASLELYSRDEQRASNVfDLEGIFREYNFQAQNIA

15 An illustrative EBV gB AD-6 nucleic acid sequence is shown as SEQ ID NO: 12.

SEQ ID NO: 12
TTCATCTCACTAAACACCTCCCTCATCGAGAACATTGACTTTGCCTCCCT
GGAGCTGTACTCACGGGACGAACAGCGTGCCTCCAACGTCTTTGACCTGG
AGGGCATCTTCCGGGAGTACAACTTCCAGGCGCAAAACATCGCC

Accordingly, in one aspect, the invention provides a human herpesvirus immunogenic peptide comprising SEQ ID NO: 7, SEQ ID NO: 9 or SEQ ID NO: 11, or a sequence having at least 75% (suitably, at least 80%, at least 85%, at least 90%, at least 95%, at least 96%, at least 97%, at least 98% or at least 99%) identity thereto, or an immunogenic fragment thereof.

In another aspect, the invention provides an isolated human herpesvirus immunogenic peptide comprising SEQ ID NO: 7, SEQ ID NO: 9 or SEQ ID NO: 11, or a sequence having at least 75% (suitably, at least 80%, at least 85%, at least 90%, at least 95%, at least 96%, at least 97%, at least 98% or at least 99%) identity thereto, or an immunogenic fragment thereof.

In some embodiments, the human herpesvirus immunogenic peptide consists of SEQ ID NO: 7, SEQ ID NO: 9 or SEQ ID NO: 11, or a sequence having at least 75% (suitably, at least 80%, at least 85%, at least 90%, at least 95%, at least 96%, at least 97%, at least 98% or at least 99%) identity thereto, or an immunogenic fragment thereof.

In one embodiment, the human herpesvirus immunogenic peptide comprises SEQ ID NO: 7, a sequence having at least 75% (suitably, at least 80%, at least 85%, at least 90%, at least 95%, at least 96%, at least 97%, at least 98% or at least 99%) identity thereto, or an immunogenic fragment thereof.

In one embodiment, the human herpesvirus immunogenic peptide comprises SEQ ID NO: 9, a sequence having at least 75% (suitably, at least 80%, at least 85%, at least 90%, at least 95%, at least 96%, at least 97%, at least 98% or at least 99%) identity thereto, or an immunogenic fragment thereof.

In one embodiment, the human herpesvirus immunogenic peptide comprises SEQ ID NO: 11, a sequence having at least 75% (suitably, at least 80%, at least 85%, at least 90%, at least 95%, at least 96%, at least 97%, at least 98% or at least 99%) identity thereto, or an immunogenic fragment thereof.

In some embodiments, the human herpesvirus immunogenic peptide comprises SEQ ID NO: 7 or a sequence having at least 75% (suitably, at least 80%, at least 85%, at least 90%, at least 95%, at least 96%, at least 97%, at least 98% or at least 99%) identity thereto.

In some embodiments, the human herpesvirus immunogenic peptide comprises SEQ ID NO: 9 or a sequence having at least 75% (suitably, at least 80%, at least 85%, at least 90%, at least 95%, at least 96%, at least 97%, at least 98% or at least 99%) identity thereto.

In some embodiments, the human herpesvirus immunogenic peptide comprises SEQ ID NO: 11 or a sequence having at least 75% (suitably, at least 80%, at least 85%, at least 90%, at least 95%, at least 96%, at least 97%, at least 98% or at least 99%) identity thereto.

The human herpesvirus immunogenic peptide or immunogenic fragment of the invention comprises at least one immunogenic peptide epitope.

The immunogenic peptide epitope induces an immune response when administered to a subject. The immunogenic peptide epitope may induce a T cell response (e.g. a cytotoxic T cell response) and/or a humoral immune response in a subject. Preferably, the immunogenic peptide epitope may induce a memory humoral immune response in a subject. Suitably, the present immunogenic peptide or immunogenic fragment thereof is capable of inducing a humoral response comprising an antibody response that is capable of reducing or inhibiting at least part of the human herpesvirus lifecycle in a host organism/tissue/cell. For example, the antibody may reduce the cell-to-cell spread of the human herpesvirus—as described herein.

Methods for determining if a peptide is immunogenic, i.e. induces an immune response, are well-known in the art and include, for example, immune cell activation assays using CD4+ and/or CD8+ T cells or B cells. Suitable markers for activation may include cell proliferation and/or signature cytokines (e.g. for T cells—IFNγ, TNFα, IL-2, IL-13 and/or IL-17) production using methods such as ELISpot. Suitably, a peptide may be assayed for the production of antibodies, for example, using the method described herein.

An immunogenic peptide epitope refers to a peptide which is recognised by either a T cell receptor (TCR) or a B cell receptor (BCR)/antibody. Suitably, the immunogenic peptide epitope is a T cell epitope or a B cell epitope.

In an adaptive immune response, T cells are capable of recognising internal epitopes of a protein antigen, e.g. of AD-6. Antigen presenting cells (APC) take up protein antigens and degrade them into short peptide fragments. A peptide may bind to a major histocompatibility complex (MHC) class II molecule within the cell and be carried to the cell surface. When presented at the cell surface in the context of an MHC molecule, the peptide may be recognised by a T cell (via the TCR), in which case the peptide is a T cell epitope.

In one embodiment, the peptide epitope is a T cell epitope. Suitably, a peptide epitope may be capable of being recognised by a TCR when presented at the cell surface in the context of a MHC molecule. Peptides which bind to MHC class II molecules are typically between 8 and 20 amino acids in length, more usually between 10 and 17 amino acids in length, and can be longer (for example up to 40 amino acids). These peptides lie in an extended conformation along the MHC II peptide-binding groove which (unlike the MHC class I peptide-binding groove) is open at both ends. The peptide is held in place mainly by main-chain atom contacts with conserved residues that line the peptide-binding groove.

Peptides that bind to MHC class I are typically 7 to 13, more usually 8 to 10 amino acids in length. The binding of the peptide is stabilised at its two ends by contacts between atoms in the main chain of the peptide and invariant sites in the peptide-binding groove of all MHC class I molecules. There are invariant sites at both ends of the groove which bind the amino and carboxy termini of the peptide. Variations in peptide length are accommodated by a kinking in the peptide backbone, often at proline or glycine residues that allow flexibility.

A T cell epitope may thus be a peptide derivable from an antigen which is capable of binding to the peptide-binding groove of an MHC molecule and being recognised by a T cell in the context of an MHC molecule.

The minimal T cell epitope is the shortest fragment derivable from a T cell epitope, which is capable of binding to the peptide-binding grove of an MHC class I or II molecule and being recognised by a T cell in the contact of an MHC molecule. For a given immunogenic region, e.g. AD-6, it is typically possible to generate a “nested set” of overlapping peptides which act as epitopes, all of which contain the minimal epitope but differ in their flanking regions.

Thus, it is possible to identify the minimal T cell epitope for a particular MHC molecule: T cell combination by measuring the response to truncated peptides, i.e. peptide fragments. For example, if a response is obtained to the peptide comprising residues 1-20 in the overlapping library, sets which are truncated at both ends (i.e. 1-19, 1-18, 1-17 etc. and 2-20, 3-20, 4-20 etc.) can be used to identify the minimal epitope.

Methods for identifying T cell epitopes within a protein or peptide sequence are known in the art and include, but are not limited to MHC binding assays and/or immune cell activation assays using CD4+ and/or CD8+ T cells. Suitable markers for activation may include cell proliferation and/or signature cytokines (e.g. for T cells—IFNγ, TNFα, IL-13 and/or IL-17) production using methods such as ELISpot.

Bioinformatics methods for predicting T cell epitopes from a protein are known in the art and include, but are not limited to, EpiDOCK, MotifScan, Rankpep, SYFPEITHI, MAPPP, PREDIVAC, PEPVAC, EPISOPT, Vaxign, MHCPred, EpiTOP, BIMAS, TEPITOPE, Propred, E[iJen, IEDB-MHCI, IEDB-MHCII, MULTIPRED2, MHC2PRED, NetMHC, NetMHCII, NetMHCpan, NetMHCIIpan, nHLApred, SVMHC, SVRMHC, NetCTL and WAPP.

In one embodiment, the peptide epitope is a B cell epitope. A B cell epitope refers to a peptide which is capable of binding to a B cell receptor (BCR)/antibody. B cell epitopes are generally divided into two categories, linear epitopes and conformational epitopes, based on their structure and interaction with the antibody. A linear epitope is formed by the 3D conformation adopted by the interaction of contiguous amino acid residues. In contrast, a conformational epitope is formed by the 3D conformation adopted by the interaction of discontiguous amino acid residues.

Suitably, the peptide epitope may be a linear B cell epitope. Suitably, the peptide epitope may be a conformational B cell epitope.

Bioinformatics methods for predicting B cell epitopes from a protein are known in the art and include, but are not limited to, linear B cell epitope predictors such as PEOPLE, BepiPred, ABCpred, LBtope, BCPREDS and SVMtrip and conformational B cell epitope predictors such as CEP, DiscoTope, ElliPro, PEPITO, SEPPA, EPITOPIA, EPSVR, EPIPRED, PEASE, MIMOX, PEPITOPE, EpiSearch, MIMOPRO and CBTOPE.

Suitably, B cell epitopes may be identified from epitopes bound by antibodies isolated from a subject previously infected/recovered from a human herpesvirus infection and/or previously vaccinated with an antigen from the human herpesvirus. Methods for determining the epitope bound by an antibody (i.e. a B cell epitope) include, but are not limited to, X-ray crystallography, cryogenic electron microscopy, array-based oligo-peptide screening, site-directed mutagenesis mapping, high-throughput mutagenesis mapping, hydrogen-deuterium exchange and cross-linking-coupled mass spectrometry.

The human herpesvirus immunogenic peptide or immunogenic fragment may comprise at least one B cell epitope and/or at least one T cell epitope as defined herein. Suitably, the human herpesvirus immunogenic peptide or immunogenic fragment may comprise at least one B cell epitope and at least one T cell epitope as defined herein.

The peptide epitope(s) may independently be at least 7, at least 10, at least 15, at least 20, at least 30, at least 40 or at least 50 amino acids in length.

The peptide epitope(s) may independently be about 7 to about 50 or about 7 to about 20 amino acids in length.

It will be appreciated by one skilled in the art that an AD-6 immunogenic peptide provided herein (e.g. an AD-6 immunogenic peptide of SEQ ID NO: 7, SEQ ID NO: 9 or SEQ ID NO: 11) may comprise additional amino acids at the N- and/or C-terminus (e.g. at the N- and/or C-terminus of SEQ ID NO: 7, SEQ ID NO: 9 or SEQ ID NO: 11). In particular, the skilled person will appreciate that an AD-6 immunogenic peptide provided herein (e.g. an AD-6 immunogenic peptide of SEQ ID NO: 7, SEQ ID NO: 9 or SEQ ID NO: 11) having a small number of amino acids added to the N- and/or C-terminus can retain the ability to produce an immune response. For example, one, two or three additional amino acids may be present at the N- and/or C-terminus of an AD-6 immunogenic peptide provided herein (e.g. an AD-6 immunogenic peptide of SEQ ID NO: 7, SEQ ID NO: 9 or SEQ ID NO: 11).

In some embodiments, the human herpesvirus immunogenic peptide is about 50 (suitably, about 49, about 48, about 47 or about 46) or fewer amino acids in length. In some preferred embodiments, the human herpesvirus immunogenic peptide is about 48 or fewer amino acids in length.

It will be appreciated by one skilled in the art that a fragment of AD-6 (e.g. a fragment of SEQ ID NO: 7, SEQ ID NO: 9 or SEQ ID NO: 11) can retain the ability to produce an immune response. Thus, in some embodiments, the human herpesvirus immunogenic peptide comprises a fragment of any one of SEQ ID NO: 7, SEQ ID NO: 9 or SEQ ID NO: 11.

In some embodiments, said immunogenic fragment is about 47 (suitably, about 45, about 40, about 35, about 30, about 25, about 20, about 19, about 18, about 17, about 16, about 15, about 14, about 13, about 12, about 11, about 10) or fewer amino acids in length. Preferably, said immunogenic fragment is about 20 (suitably, about 19, about 18, about 17, about 16, about 15, about 14, about 13, about 12, about 11, about 10) or fewer amino acids in length. More preferably, said immunogenic fragment is about 15 (suitably, about 14, about 13, about 12, about 11, about 10) or fewer amino acids in length.

Illustrative immunogenic fragments of HCMV gB AD-6 are shown below:

(SEQ ID NO: 13)
VDSMIALDIDPLENT
(SEQ ID NO: 14)
IALDIDPLENTDFRV
(SEQ ID NO: 15)
IDPLENTDFRVLELY
(SEQ ID NO: 16)
ENTDFRVLELYSQKE
(SEQ ID NO: 17)
FRVLELYSQKELRSS
(SEQ ID NO: 18)
ELYSQKELRSSNVFD
(SEQ ID NO: 19)
QKELRSSNVFDLEEI
(SEQ ID NO: 20)
RSSNVFDLEEIMREF
(SEQ ID NO: 21)
VFDLEEIMREFNSYK
(SEQ ID NO: 22)
EEIMREFNSYKQRVK
(SEQ ID NO: 23)
REFNSYKQRVKYVED
(SEQ ID NO: 24)
SYKQRVKYVEDKVVD
(SEQ ID NO: 25)
ELYSQKELRSS
(SEQ ID NO: 26)
SYKQRVKYVED

In some embodiments, said immunogenic fragment is selected from SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25 and/or SEQ ID NO: 26.

In some embodiments, said immunogenic fragment is selected from SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25 or SEQ ID NO: 26.

In some preferred embodiments, said immunogenic fragment is SEQ ID NO: 25 and/or SEQ ID NO: 26.

Preferably, said immunogenic fragment is SEQ ID NO: 25.

As used herein, the term “immunogenic fragment” means a fragment of the specified sequence which retains the ability to induce an immune response.

It will be appreciated by one skilled in the art that a variant of any one of SEQ ID NO: 13 to SEQ ID NO: 26 can retain the ability to produce an immune response. The variant may have one, two or three amino acid substitutions. Thus, in some embodiments, the human herpesvirus immunogenic peptide comprises a variant of any one of SEQ ID NO: 13 to SEQ ID NO: 26 having one, two or three amino acid substitutions. Suitably, the human herpesvirus immunogenic peptide comprises a variant of SEQ ID NO: 25 or SEQ ID NO: 26 having one, two or three amino acid substitutions. Suitably, the human herpesvirus immunogenic peptide comprises a variant of SEQ ID NO: 25 having one, two or three amino acid substitutions.

It will be appreciated by one skilled in the art that a shorter fragment of SEQ ID NO: 7 than the immunogenic fragments set forth in SEQ ID NO: 13 to SEQ ID NO: 26 can retain the ability to produce an immune response. Thus, in some embodiments, the human herpesvirus immunogenic peptide comprises a variant of any one of SEQ ID NO: 13 to SEQ ID NO: 26, having one, two or three amino acid deletions at the N- or C-terminus. Suitably, the human herpesvirus immunogenic peptide comprises a variant of SEQ ID NO: 25 or SEQ ID NO: 26 having one, two or three amino acid deletions at the N- or C-terminus. Suitably, the human herpesvirus immunogenic peptide comprises a variant of SEQ ID NO: 25 having one, two or three amino acid deletions at the N- or C-terminus.

It will be appreciated by one skilled in the art that a variant which is also a shorter fragment of SEQ ID NO: 7 than the immunogenic fragments set forth in SEQ ID NO: 13 to SEQ ID NO: 26 can retain the ability to produce an immune response. Thus, in some embodiments, the human herpesvirus immunogenic peptide comprises a variant of any one of SEQ ID NO: 13 to SEQ ID NO: 26, having one, two or three amino acid substitutions and having one, two or three amino acid deletions at the N- or C-terminus. Suitably, the human herpesvirus immunogenic peptide comprises a variant of SEQ ID NO: 25 or SEQ ID NO: 26 having one, two or three amino acid substitutions, and having one, two or three amino acid deletions at the N- or C-terminus. Suitably, the human herpesvirus immunogenic peptide comprises a variant of SEQ ID NO: 25 having one, two or three amino acid substitutions, and having one, two or three amino acid deletions at the N- or C-terminus.

Suitably, whether a peptide retains the ability to produce an immune response may be determined using the methods for determining if a peptide is immunogenic as described herein.

In some embodiments, said immunogenic fragment is selected from SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, or a variant thereof.

In some preferred embodiments, said immunogenic fragment is SEQ ID NO: 25 or SEQ ID NO: 26, or a variant thereof.

In some preferred embodiments, said immunogenic fragment is SEQ ID NO: 25 or SEQ ID NO: 26, or a variant thereof.

In some preferred embodiments, said immunogenic fragment is SEQ ID NO: 25 or SEQ ID NO: 26.

In some embodiments, said immunogenic fragment is SEQ ID NO: 13 or a variant thereof.

In some embodiments, said immunogenic fragment is SEQ ID NO: 14 or a variant thereof.

In some embodiments, said immunogenic fragment is SEQ ID NO: 15 or a variant thereof.

In some embodiments, said immunogenic fragment is SEQ ID NO: 16 or a variant thereof.

In some embodiments, said immunogenic fragment is SEQ ID NO: 17, or a variant thereof.

In some embodiments, said immunogenic fragment is SEQ ID NO: 18, or a variant thereof.

In some embodiments, said immunogenic fragment is SEQ ID NO: 19, or a variant thereof.

In some embodiments, said immunogenic fragment is SEQ ID NO: 20, or a variant thereof.

In some embodiments, said immunogenic fragment is SEQ ID NO: 21, or a variant thereof.

In some embodiments, said immunogenic fragment is SEQ ID NO: 22, or a variant thereof.

In some embodiments, said immunogenic fragment is SEQ ID NO: 23, or a variant thereof.

In some embodiments, said immunogenic fragment is SEQ ID NO: 24, or a variant thereof.

In some embodiments, said immunogenic fragment is SEQ ID NO: 25, or a variant thereof.

In some embodiments, said immunogenic fragment is SEQ ID NO: 26, or a variant thereof.

The variant may have one, two or three amino acid substitutions and/or may have one, two or three amino acid deletions at the N- and/or C-terminus. Suitably, the variant may have one, two or three amino acid substitutions and one, two or three amino acid deletions at the N- and/or C-terminus.

The variant may have one, two or three amino acid substitutions.

The variant may have one, two or three amino acid deletions at the N- and/or C-terminus. Suitably, the variant may have one, two or three amino acid deletions at the N- and C-terminus. Suitably, the variant may have one, two or three amino acid deletions at the N-terminus. Suitably, the variant may have one, two or three amino acid deletions at the C-terminus.

In some preferred embodiments, said amino acid substitutions are conservative amino acid substitutions.

Amino acids with similar biochemical properties may be defined as amino acids which can be substituted via a conservative substitution.

Conservative amino acid substitutions may be made on the basis of similarity in polarity, charge, solubility, hydrophobicity, hydrophilicity, and/or the amphipathic nature of the residues. For example, negatively charged amino acids include aspartic acid and glutamic acid; positively charged amino acids include lysine and arginine; and amino acids with uncharged polar head groups having similar hydrophilicity values include leucine, isoleucine, valine, glycine, alanine, asparagine, glutamine, serine, threonine, phenylalanine, and tyrosine.

Conservative substitutions may be made, for example according to Table 1 below. Amino acids in the same block in the second column and preferably in the same line in the third column may be substituted for each other:

TABLE 1

| | | |
|---|---|---|
| ALIPHATIC | Non-polar | G A P |
| | | I L V |
| | Polar-uncharged | C S T M |
| | | N Q |
| | Polar-charged | D E |
| | | K R |
| AROMATIC | | H F W Y |

The present invention also encompasses homologous substitution (substitution and replacement are both used herein to mean the interchange of an existing amino acid residue, with an alternative residue) i.e. like-for-like substitution such as basic for basic, acidic for acidic, polar for polar etc.

An aliphatic, non-polar amino acid may be a glycine, alanine, proline, isoleucine, leucine or valine residue.

An aliphatic, polar uncharged amino may be a cysteine, serine, threonine, methionine, asparagine or glutamine residue.

An aliphatic, polar charged amino acid may be an aspartic acid, glutamic acid, lysine or arginine residue.

An aromatic amino acid may be a histidine, phenylalanine, tryptophan or tyrosine residue.

Suitably, a conservative substitution may be made between amino acids in the same line in Table 1.

In some embodiments, said human herpesvirus is HCMV, HSV-1, and/or EBV.

In one preferred embodiment, said human herpesvirus is HCMV.

In one embodiment, said human herpesvirus is HSV-1.

In one embodiment, said human herpesvirus is EBV.

### Nucleic Acid Sequence

In a further aspect, the present invention provides a nucleic acid sequence encoding a human herpesvirus immunogenic peptide of the invention.

In some embodiments, said nucleic acid sequence is selected from SEQ ID NO: 8, SEQ ID NO: 10 and SEQ ID NO: 12.

In one embodiment, said nucleic acid sequence is SEQ ID NO: 8.

In one embodiment, said nucleic acid sequence is SEQ ID NO: 10.

In one embodiment, said nucleic acid sequence is SEQ ID NO: 12.

The term “nucleic acid sequence” in relation to the present invention can be a double stranded or single stranded molecule and includes genomic DNA, cDNA, synthetic DNA, RNA and a chimeric DNA/RNA molecule. Preferably, it means a DNA sequence encoding a human herpesvirus immunogenic peptide of the present invention.

Typically, the nucleic acid sequence encompassed by the scope of the present invention is prepared using recombinant DNA techniques (i.e. recombinant DNA), as described herein.

In a preferred embodiment, the nucleic acid sequence encoding a human herpesvirus immunogenic peptide of the invention is an expression cassette.

In a further aspect, the present invention provides a vector comprising a nucleic acid sequence of the invention.

A vector is a tool that allows or facilitates the transfer of an entity from one environment to another. In accordance with the present invention, and by way of example, some vectors used in recombinant nucleic acid techniques allow entities, such as a nucleic acid of the invention, to be transferred into and expressed by a target cell. The vector may facilitate the integration of the nucleic acid sequence of the invention to maintain the nucleic acid sequence of the invention and its expression within the target cell.

The vector may be or may include an expression cassette (also termed an expression construct). Expression cassettes as described herein comprise regions of nucleic acid containing sequences capable of being transcribed. Thus, sequences encoding mRNA, tRNA and rRNA are included within this definition.

The term “cassette”—which is synonymous with “construct”—includes a nucleic acid sequence directly or indirectly attached to a promoter. The expression cassettes for use in the invention comprise a promoter for the expression of the nucleic acid sequence of the invention. Preferably, the cassette comprises at least a nucleic acid sequence of the invention operably linked to a promoter. The choice of expression cassette, e.g. plasmid or viral vector, will often depend on the host cell into which it is to be introduced.

The vector may contain one or more selectable marker genes (e.g. a kanamycin resistance gene) and/or traceable marker gene(s) (e.g. a gene encoding green fluorescent protein (GFP)).

### Inhibitor

Herpesviruses grow in vivo both as a cell free and cell associated virus. gB is present in the virion and virally infected cells express gB at the cell surface. Thus, an inhibitor of gB may bind to gB in the virion or on the surface of infected cells, i.e. the inhibitor (e.g. an antibody) may recognise infected cells.

Accordingly, in a further aspect, the invention provides an inhibitor of human herpesvirus glycoprotein-B which is capable of binding to human herpesvirus glycoprotein-B on the surface of a cell.

In a further aspect, the invention provides an inhibitor of human herpesvirus glycoprotein-B which is capable of binding to human herpesvirus glycoprotein-B within SEQ ID NO: 7, SEQ ID NO: 9 or SEQ ID NO: 11, or a sequence having at least 75% identity thereto, or a fragment thereof.

Binding of gB on the surface of a cell may be determined using a method known in the art, including but not limited to cell-based binding assays using FACS or surface plasmon resonance. Suitably, binding of gB on the cell surface may be performed using the assay described herein (see Example 2). Suitably, the method may employ HFF cells infected with HCMV. After 72 hours, cells are stained with the AD-6 antibody and then incubated with fluorochrome-conjugated secondary antibody that recognises the AD-6 antibody. This assay can be performed in any cell type that supports lytic HCMV replication (e.g. HFF, ARPE, U373, dendritic cell, macrophage, endothelial cell).

In some preferred embodiments, the inhibitor binds to said human herpesvirus gB within a human herpesvirus immunogenic peptide as described herein. Preferably, the inhibitor may be capable of binding to an immunogenic peptide epitope within a human herpesvirus immunogenic peptide as described herein.

The inhibitor may be selected from one or more of the following: an antibody, an Ig fusion protein, a polypeptide, a peptide, a polynucleotide, a small molecule, a non-antibody scaffold, an aptamer, or combinations thereof.

The inhibitor may reduce or inhibit at least part of the human herpesvirus lifecycle in a host organism/tissue/cell. For example, the inhibitor may reduce the cell-to-cell spread of the human herpesvirus. Models of cell-to-cell spread are known in the art. For example, a cell line (e.g. human fibroblast or epithelial cell line) may be infected with a human herpesvirus and treated with the inhibitor and complement. A reduction in cell-to-cell spread may be determined by a reduction of cell infection (which is measured by enumerating the % of infected cells) compared to a control experiment which has not been treated with inhibitor.

The inhibitor may reduce viral cell-to-cell spread by at least 1.5-fold (suitably, at least 2-fold, at least 2.5-fold, at least 3-fold, at least 4-fold or at least 5-fold) relative to viral cell-to-cell spread in the absence of inhibitor.

The inhibitor may reduce viral cell-to-cell spread by at least 10% (suitably, at least 20%, at least 30%, at least 40%, at least 50% or at least 100%) relative to viral cell-to-cell spread in the absence of inhibitor. Preferably, the inhibitor may reduce viral cell-to-cell spread by at least 20% relative to viral cell-to-cell spread in the absence of the immunogenic composition. More preferably, the inhibitor may reduce viral cell-to-cell spread by at least 30% relative to viral cell-to-cell spread in the absence of inhibitor.

The inhibitor (e.g. an antibody) may be capable of selectively binding to a human herpesvirus gB (e.g. a human herpesvirus immunogenic peptide as described herein) and thus may have a greater binding affinity for a human herpesvirus gB as compared to its binding affinity for other proteins/molecules. Preferably, the inhibitor does not bind to other proteins or binds with a greatly reduced affinity compared to the binding to said human herpesvirus gB (e.g. with an affinity of at least 10, 50, 100, 500, 1000 or 10000 times less than its affinity for said human herpesvirus gB). Thus, the inhibitor as referred to herein may bind to said human herpesvirus gB (e.g. a human herpesvirus immunogenic peptide as described herein) with at least 10, 50, 100, 500, 1000 or 10000 times the affinity of its binding to other proteins. The binding affinity of the inhibitor can be determined using methods well known in the art, such as with the Biacore system.

The inhibitor may have a high binding affinity for said human herpesvirus gB (e.g. a human herpesvirus immunogenic peptide as described herein), i.e. may have a K_{d} in the range of 10⁻⁵ M, 10⁻⁶ M, 10⁻⁷ M or 10⁻⁹ M or less. The anti-gB binding domain of the inhibitor may have a binding affinity for said human herpesvirus gB (e.g. a human herpesvirus immunogenic peptide as described herein) that corresponds to a K_{d} of less than 30 nM, 20 nM, 15 nM or 10 nM, more preferably of less than 10, 9.5, 9, 8.5, 8, 7.5, 7, 6.5, 6, 5.5, 5, 4.5, 4, 3.5, 3, 2.5, 2, 1.5 or 1 nM, most preferably less than 0.9, 0.8, 0.7, 0.6, 0.5, 0.4, 0.3, 0.2 or 0.1 nM.

Any appropriate method of determining K_{d} may be used. Preferably, the K_{d} is determined by testing various concentrations of the test agent against various concentrations of antigen (i.e. human herpesvirus gB, e.g. a human herpesvirus immunogenic peptide as described herein) in vitro to establish a saturation curve, for example using the Lineweaver-Burk method, or by using commercially available binding model software, such as the 1:1 binding model in the BIAcore 1000 Evaluation software.

In some embodiments, said inhibitor is an antibody.

In some embodiments, said antibody is a monoclonal antibody, a humanised antibody, a single-chain antibody or an antibody fragment. Suitably antibodies include, but are not limited to, monoclonal and polyclonal antibodies, engineered antibodies including chimeric, CDR-grafted and humanised antibodies, single-chain antibodies, antibody fragments and artificially selected antibodies produced using phage display or alternative techniques.

Suitably the antibody is a monoclonal antibody.

Suitable antibody fragments capable of binding to a selected target, include Fv, ScFv, F(ab′) and F(ab′)₂. In addition, alternatives to classical antibodies may also be used in the invention, for example “avibodies”, “avimers”, “anticalins”, “nanobodies” and “DARPins”.

Reference to “scFv” or “single-chain variable fragment” as used herein includes molecules wherein the variable heavy (VH) and variable light chain (VL) of an antibody are linked via a flexible oligopeptide. A scFv is thus a fusion between at least one variable heavy and at least one variable light chain.

Reference to a “complementarity determining region” or “CDR” as used herein refers to the regions of hypervariability within antibodies which bind to the specific antigen e.g. to CLEC14A. The CDRs of an antibody thus usually provide an antibody with its binding specificity. Three CDRs may be present in the variable region of each heavy chain of an intact antibody molecule (i.e. comprising two full length heavy and two full length light chains) and three CDRs may be present in the variable region of each light chain (heavy chain CDRs 1, 2 and 3 and light chain CDRs 1, 2 and 3, numbered from the amino to the carboxy terminus). The CDRs of the variable regions of a heavy and light chain of an antibody can be predicted from the heavy and light chain variable region sequences of the antibody, using prediction software known in the art, including but not limited to the Abysis algorithm (www.bioinf.org.uk/abysis/sequence_input/key_annotation/key_annotation.cgi) and IMGT/V-QUEST software, e.g. the IMGT algorithm (ImMunoGeneTics) (www.IMGT.org; Lefranc et al, 2009 NAR 37:D1006-D1012; and Lefranc 2003, Leukemia 17:260-266). CDRs may vary in length, depending on the antibody from which they are predicted and between the heavy and light chains. A CDR for example, may range from 2 amino acids in length to 20 amino acids in length, e.g. from 3-14 amino acids in length. The Kabat nomenclature is followed herein in order to define the positioning of the CDRs (Kabat et al., 1991, 5^{th} Ed. Public Health Service, National Institutes of Health, Bethesda, MD, 647-669, incorporated herein by reference).

The term “heavy chain variable region” (VH domain) as used herein refers to the variable region of a heavy chain of an antibody molecule.

The term “light chain variable region” (VL domain) as used herein refers to the variable region of a light chain of an antibody molecule.

Antibodies capable of binding to human herpesvirus gB (e.g. within a human herpesvirus immunogenic peptide as described herein) can be produced using any method known in the art. Methods for the production of monoclonal antibodies, recombinant antibodies and aptamers are reviewed in Groff et al. (Biotechnology Advances 2015, 33(8):1787-1798) and Dangi et al. (Front Pharmacol. 2018; 9:630)—each of which is incorporated herein by reference.

Antibodies capable of inhibiting gB may be identified using any method known in the art, including an assay for binding of gB on the surface of a cell or a viral cell-to-cell spread assay as described herein.

### Immunogenic Composition and Pharmaceutical Composition

In a further aspect, the invention provides an immunogenic composition comprising a human herpesvirus immunogenic peptide of the invention.

In a further aspect, the invention provides an immunogenic composition comprising a nucleic acid sequence of the invention or a vector of the invention.

In a further aspect, the invention provides a pharmaceutical composition comprising an inhibitor of the invention.

The immunogenic composition may induce an immune response when administered to a subject. The immunogenic composition may induce a T cell response (e.g. a cytotoxic T cell response) and/or a humoral immune response in a subject. Preferably, the immunogenic composition may induce a memory humoral immune response in a subject. Suitably, the present immunogenic composition is capable of inducing a humoral response comprising an antibody response that is capable of reducing or inhibiting at least part of the human herpesvirus lifecycle in a host organism/tissue/cell. For example, the antibody may reduce the cell-to-cell spread of the human herpesvirus—as described herein.

Methods for determining if an immunogenic composition induces an immune response are well-known in the art and include, for example, immune cell activation assays using CD4⁺ and/or CD8⁺ T cells or B cells as described herein above.

Suitably, the induction of humoral immunity to a human herpesvirus may refer to B cell activity. The activity of B cells may be determined using methods which are known in the art. For example, the activity of B cells may be determined by determining antibody (e.g. IgG or IgM) production or antiviral cytokine production (e.g. IFNγ or IL-6) following treatment with the immunogenic composition. Enhancement of B cell activity may be determined by an increase in antibody or effector cytokine (e.g. percentage of effector cytokine positive cells) as determined by flow cytometry or amount of effector cytokine mRNA or protein as determined by qPCR, ELISA or ELISPOT, respectively) compared to a control experiment which has not been treated with the immunogenic composition.

For example, levels of antiviral cytokine production in B-cells treated with an immunogenic composition may be increased by at least 10%, at least 20%, at least 30%, at least 40%, at least 50%, at least 75% or at least 100% compared to control cells which have not been treated with the immunogenic composition.

Suitably, the induction of T cell immunity (e.g. of humoral immunity) to a human herpesvirus may refer to CD4⁺ T cell activity. The activity of CD4⁺ T cells may be determined using methods which are known in the art. For example, CD4⁺ T cell activity may be assessed by determining effector cytokine production (i.e. IFNγ, IL-21 or IL-4) following treatment with the immunogenic composition. Enhancement of CD4⁺ T cell activity may be determined by an increase in effector cytokine (e.g. percentage of effector cytokine positive cells as determined by flow cytometry or amount of effector cytokine mRNA or protein as determined by qPCR, ELISA or ELISPOT, respectively) compared to a control experiment which has not been treated with the immunogenic composition.

For example, levels of effector cytokine in CD4⁺ T cell treated with an immunogenic composition may be increased by at least 10%, at least 20%, at least 30%, at least 40%, at least 50%, at least 75% or at least 100% compared to control cells which have not been treated with the immunogenic composition.

Suitably, induced T cell immunity to a human herpesvirus may refer to CD8+ T cell activity. The activity of CD8⁺ T cells may be determined using methods which are known in the art. For example, CD8⁺ T cell activity may be assessed by determining effector cytokine production (i.e. IFNγ/TNF/IL-2) following treatment with the immunogenic composition. Enhancement of CD8⁺ T cell activity may be determined by an increase in effector cytokine (e.g. percentage of effector cytokine positive cells as determined by flow cytometry or amount of effector cytokine mRNA or protein as determined by qPCR or ELISA or ELISPOT, respectively) compared to a control experiment which has not been treated with the immunogenic composition. Proliferative expansion of antigen-specific CD8⁺ T cells can also be assessed by staining with MHC/peptide multimers.

For example, levels of effector cytokine in CD8⁺ T cells treated with an immunogenic composition may be increased by at least 10%, at least 20%, at least 30%, at least 40%, at least 50%, at least 75% or at least 100% compared to control cells which have not been treated with the immunogenic composition.

The pharmaceutical composition may reduce or inhibit at least part of the human herpesvirus lifecycle in a host organism/tissue/cell. For example, the pharmaceutical composition may reduce the cell-to-cell spread of the human herpesvirus. Models of cell-to-cell spread are known in the art and described herein above.

The pharmaceutical composition may reduce viral cell-to-cell spread by at least 1-fold (suitably, at least 1.5-fold, at least 2-fold, at least 2.5-fold, at least 3-fold, at least 4-fold or at least 5-fold) relative to viral cell-to-cell spread in the absence of the immunogenic composition. The immunogenic composition may reduce viral cell-to-cell spread by at least 10% (suitably, at least 20%, at least 30%, at least 40%, at least 50% or at least 100%) relative to viral cell-to-cell spread in the absence of the immunogenic composition. Preferably, the immunogenic composition may reduce viral cell-to-cell spread by at least 20% relative to viral cell-to-cell spread in the absence of the immunogenic composition. More preferably, the immunogenic composition may reduce viral cell-to-cell spread by at least 30% relative to viral cell-to-cell spread in the absence of the immunogenic composition.

In the context of the present invention, an immunogenic composition is administered to a subject in order to enhance the subject's immune response to human herpesvirus infection. The subject may be seronegative or seropositive for a human herpesvirus. Preferably, the subject is seronegative for a human herpesvirus.

In the context of the present invention, a pharmaceutical composition is administered to a subject in order to treat and/or prevent a human herpesvirus infection. The subject may be seronegative or seropositive for a human herpesvirus.

Suitably, the immunogenic composition or pharmaceutical composition will be formulated for administration by injection, for example by intramuscular, intradermal, intravenous or sub-cutaneous injection.

An immunogenic composition or a pharmaceutical composition will generally be administered in admixture with a pharmaceutical carrier, excipient or diluent, particularly for human therapy. Suitably, an immunogenic composition or a pharmaceutical composition may be administered in admixture with an adjuvant, particularly for human therapy.

### Vaccine

In a further aspect, the invention provides a vaccine comprising a human herpesvirus immunogenic peptide of the invention.

In a further aspect, the invention provides a vaccine comprising a nucleic acid sequence of the invention or a vector of the invention.

In a further aspect, the invention provides a vaccine comprising an inhibitor of the invention.

The vaccine may induce a protective immune response when administered to a subject, i.e. an immune response that is protective against subsequent challenge with a human herpesvirus. The vaccine may induce a protective T cell response (e.g. a cytotoxic T cell response) and/or a protective humoral immune response in a subject. Preferably, the vaccine may induce a memory humoral immune response in a subject. Suitably, the present vaccine is capable of inducing a humoral response comprising an antibody response that is capable of reducing or inhibiting at least part of the human herpesvirus lifecycle in a host organism/tissue/cell. For example, the antibody may reduce the cell-to-cell spread of the human herpesvirus—as described herein.

Methods for determining if a vaccine induces a protective immune response are well-known in the art and include, for example, vaccination of a subject and subsequent challenge (either natural or deliberate) with a human herpesvirus.

In the context of the present invention, a vaccine is administered to a subject in order to enhance the subject's immune response to human herpesvirus infection. The subject may be seronegative or seropositive for a human herpesvirus. Preferably, the subject is seronegative for a human herpesvirus. The subject may be seropositive for a human herpesvirus.

Suitably, the vaccine will be formulated for administration by injection, for example by intramuscular, intradermal, intravenous or sub-cutaneous injection.

A vaccine will generally be administered in admixture with a pharmaceutical carrier, excipient or diluent, particularly for human therapy. Suitably, a vaccine may be administered in admixture with an adjuvant, particularly for human therapy.

### Treatment or Prevention of Human Herpesvirus Infection

Original antigenic sin means that the immune system does not make new antibody responses if it is presented with an antigen that contains epitopes it has already seen; instead it boosts what it already knows. A human herpesvirus immunogenic peptide of the invention that presents AD-6 to the immune system (e.g. in the context of an AD-6 subunit vaccine) has the potential to be effective, particularly if the presentation of other ADs of gB is absent. This would allow the immune system to focus on generating humoral responses against the novel AD-6 epitope rather than potential “decoy” epitopes (other ADs e.g. AD-1, AD-2, AD-3, AD-4 and/or AD-5) present in gB, i.e. to avoid the issue of original ‘antigenic sin’ that is seen in HCMV seropositive patients with the current gB vaccine, i.e. the candidate gB vaccine comprising recombinant HCMV gB and MF59 adjuvant (Baraniak et al. 2019 J. Infect. Dis. 220(2) 228-232). In other words, this would enable the vaccination of individuals who are already infected (e.g. with a herpesvirus such as HCMV) as well as uninfected individuals. This ability is important for the treatment of herpesvirus infection, and particular HCMV infection, due to the prevalence of these infections, with approximately 50% of the world's population already seropositive for HCMV and at risk from disease caused by reactivation.

Thus, the presentation of an AD-6 immunogenic peptide as described herein (e.g. in the context of an AD-6 subunit vaccine) to the immune system would both focus the humoral response against AD-6 and prevent deleterious response against other gB domains. This may be sufficient to improve the efficacy of a gB-based vaccine, for example, in both seropositive and seronegative individuals.

In a further aspect, the invention provides an immunogenic composition of the invention for use in the treatment or prevention of human herpesvirus infection in a subject.

In a further aspect, the invention provides a pharmaceutical composition of the invention for use in the treatment or prevention of human herpesvirus infection in a subject.

In a further aspect, the invention provides a vaccine of the invention for use in the treatment or prevention of human herpesvirus infection in a subject.

In a further aspect, the invention provides an inhibitor of the invention for use in the treatment or prevention of human herpesvirus infection in a subject.

Also provided is a method for the treatment and/or prevention of a human herpesvirus infection, comprising administering an immunogenic composition of the invention, a pharmaceutical composition of the invention, a vaccine of the invention or an inhibitor of the invention to a subject in need thereof.

Also provided is a method for the treatment of a human herpesvirus infection, comprising administering an immunogenic composition of the invention, a pharmaceutical composition of the invention, a vaccine of the invention or an inhibitor of the invention to a subject in need thereof.

Also provided is a method for the prevention of a human herpesvirus infection, comprising administering an immunogenic composition of the invention, a pharmaceutical composition of the invention, a vaccine of the invention or an inhibitor of the invention to a subject in need thereof.

In some embodiments, the cell-to-cell spread of the human herpesvirus is inhibited.

In some embodiments, the immunogenic composition, pharmaceutical composition, vaccine or inhibitor is for use in the prevention of human herpesvirus infection in the subject.

In some embodiments, the immunogenic composition, pharmaceutical composition, vaccine or inhibitor is for use in the treatment of human herpesvirus infection in the subject.

In some embodiments, the human herpesvirus is HCMV, HSV-1, and/or EBV.

In one preferred embodiment, the human herpesvirus is HCMV.

In some embodiments, the human herpesvirus is HSV-1.

In some embodiments, the human herpesvirus is EBV.

The term “treat/treatment/treating” may refer generally to administering an immunogenic composition, vaccine, inhibitor or pharmaceutical composition of the invention to a subject having an existing disease or condition in order to reduce, alleviate or eliminate one or more symptoms associated with the disease, disorder or condition which is being treated and/or to slow down, reduce or block the progression of the disease, disorder or condition which is being treated.

The term “prevent/prevention/prophylaxis” may refer generally to administering an immunogenic composition, vaccine, inhibitor or pharmaceutical composition of the invention to a subject having an existing disease or condition or at risk of developing a disease or condition in order to delay or prevent the onset of the symptoms of the disease, disorder or condition. Prevention may be absolute (such that no disease occurs) or may be effective only in some individuals or for a limited amount of time.

### Combinations

The immunogenic composition of the invention, pharmaceutical composition of the invention, vaccine of the invention or inhibitor of the invention may be administered to the subject in combination with at least one further pharmaceutically active agent.

In one embodiment, the immunogenic composition of the invention may be administered to the subject in combination with at least one further pharmaceutically active agent.

In one embodiment, the pharmaceutical composition of the invention may be administered to the subject in combination with at least one further pharmaceutically active agent.

In one embodiment, the vaccine of the invention may be administered to the subject in combination with at least one further pharmaceutically active agent.

In one embodiment, the inhibitor of the invention may be administered to the subject in combination with at least one further pharmaceutically active agent.

Suitably, “in combination” may mean that the immunogenic composition, pharmaceutical composition, vaccine or inhibitor of the invention and the further pharmaceutically active agent are administered to the subject in a simultaneous, combined, sequential or separate manner.

By “simultaneous”, it is to be understood that the two agents are administered concurrently, whereas the term “combined” is used to mean they are administered, if not simultaneously, then “sequentially” within a time frame that they both are available to act therapeutically within the same time frame. Thus, administration “sequentially” may permit one agent to be administered within 5 minutes, 10 minutes or a matter of hours after the other provided the circulatory half-life of the first administered agent is such that they are both concurrently present in therapeutically effective amounts. The time delay between administration of the components will vary depending on the exact nature of the components, the interaction there-between, and their respective half-lives.

In contrast to “combined” or “sequential”, “separate” may be understood as meaning that the gap between administering one agent and the other agent is significant, i.e. the first administered agent may no longer be present in the bloodstream in a therapeutically effective amount when the second agent is administered.

Suitably, the immunogenic composition, pharmaceutical composition, vaccine or inhibitor of the invention and further pharmaceutically active agent are administered in a simultaneous, combined, or sequential manner to a subject.

Suitably, the immunogenic composition, pharmaceutical composition, vaccine or inhibitor of the invention and further pharmaceutically active agent may be administered as a single composition. For example, the immunogenic composition may comprise a nucleic acid construct encoding a human herpesvirus immunogenic peptide—as described herein—and be provided as part of a vaccine as described herein.

Suitably, the immunogenic composition, pharmaceutical composition, vaccine or inhibitor of the invention may be administered as a separate composition to the further pharmaceutically active agent. For example, the inhibitor may be a small molecule administered orally and the further pharmaceutically active agent may be a HCMV vaccine administered by intramuscular, subcutaneous or intradermal or intravenous injection. Alternatively, the inhibitor may be a small molecule administered orally or an antibody administered by intramuscular, subcutaneous or intradermal or intravenous injection and the further pharmaceutically active agent may be an antiviral (e.g. GCV) administered by intravenous injection.

The at least one further pharmaceutically active agent may be selected from an antiviral, a herpesvirus vaccine, an immunostimulatory cytokine, a checkpoint inhibitor, TCR-gene-engineered T cells, activators of innate immunity, monoclonal or bispecific antibodies, CAR cells, soluble T cell receptors, or any combination thereof.

Suitably, the at least one further pharmaceutically active agent may be selected from an antiviral, a herpesvirus vaccine, an immunostimulatory cytokine, TCR-gene-engineered T cells, activators of innate immunity, monoclonal or bispecific antibodies, CAR cells, soluble T cell receptors, or any combination thereof.

Antivirals include, but are not limited to, antivirals that target the viral DNA polymerase. Further antivirals include agents which interfere or inhibit the ability of the herpesvirus to enter host cells and others such as capsid assembly inhibitors, anti-HCMV siRNA, etc. Suitable antivirals include ganciclovir (GCV), foscavir, foscarnet, cidofovir, acyclovir (Zovirax), famciclovir (Famvir), valacyclovir (Valtrex), penciclovir, maribavir, letermovir.

Current antivirals are generally not considered to be suitable for all patients, e.g. pregnant women or neonates, because of the level of toxicity.

Suitable immunostimulatory cytokines include, but are not limited to, IFN-α, pegylated IFN-α, IFN-λ, IFNγ, GM-CSF, IL-7, IL-12, IL-15, IL-18 and IL-21.

Suitable checkpoint inhibitors include, but are not limited to, both inhibitory and activatory molecules, and interventions may apply to either or both types of molecule. Immune checkpoint inhibitors include, but are not limited to, PD-1 inhibitors, PD-L1 inhibitors, Lag-3 inhibitors, Tim-3 inhibitors, TIGIT inhibitors, BTLA inhibitors and CTLA-4 inhibitors, for example. Co-stimulatory antibodies deliver positive signals through immune-regulatory receptors including but not limited to ICOS, CD137, CD27 OX-40 and GITR.

Suitably, the checkpoint inhibitor may be a PD-1 or PD-L1 inhibitor.

Examples of suitable immune checkpoint interventions which prevent, reduce or minimize the inhibition of immune cell activity include pembrolizumab, nivolumab, atezolizumab, durvalumab, avelumab, tremelimumab and ipilimumab.

A TCR-gene-engineered cell refers to a cell, preferably a T cell, which has been engineered to introduce an exogenous nucleic acid encoding an exogenous TCR into the cell and thus redirect the specificity of cell to a target antigen of interest. In the context of the present invention, the cell is suitably redirected to a human herpesvirus target antigen of interest. TCR-gene-engineered cells and anti-HCMV T cells are described in the art, for example see Schub et al. (J Immunol 2009; 183:6819-6830) and Wagner et al. (J Biol. Chem. 2019; 294:5790-5804); each of which is incorporated herein by reference.

The present invention also encompasses administering an immunogenic composition, a pharmaceutical composition, a vaccine or an inhibitor of the invention in combination with human herpesvirus-specific (e.g. HCMV-specific) T cells which have been isolated, and preferably enriched, from a subject. The subject is suitably the same subject with human herpesvirus infection who is to be treated according to the invention.

Soluble T cell receptors (sTCR) typically comprise the variable domain and at least part of the TCR constant domain but lack the transmembrane domain and intracellular, cytoplasmic domain. Suitably, the sTCR does not comprise a transmembrane domain. Suitably, the sTCR is not anchored on the surface of cell. In the context of the present invention, the sTCR is directed to a human herpesvirus target antigen of interest.

A classical CAR is a chimeric type I trans-membrane protein which connects an extracellular antigen-recognizing domain (binder) to an intracellular signalling domain (endodomain). The binder is typically a single-chain variable fragment (scFv) derived from a monoclonal antibody (mAb), but it can be based on other formats which comprise an antibody-like antigen binding site. A spacer domain is usually necessary to isolate the binder from the membrane and to allow it a suitable orientation. A common spacer domain used is the Fc of IgG1. More compact spacers can suffice e.g. the stalk from CD8α and even just the IgG1 hinge alone, depending on the antigen. A trans-membrane domain anchors the protein in the cell membrane and connects the spacer to the endodomain.

When the CAR binds the target-antigen, this results in the transmission of an activating signal to the T-cell it is expressed on. Thus, the CAR directs the specificity and cytotoxicity of the T cell towards cells expressing the targeted antigen.

CARs typically therefore comprise: (i) an antigen-binding domain; (ii) a spacer; (iii) a transmembrane domain; and (iii) an intracellular domain which comprises or associates with a signalling domain.

The antigen binding domain is the portion of the CAR which recognizes antigen. Numerous antigen-binding domains are known in the art, including those based on the antigen binding site of an antibody, antibody mimetics, and T-cell receptors. For example, the antigen-binding domain may comprise: a single-chain variable fragment (scFv) derived from a monoclonal antibody; a natural ligand of the target antigen; a peptide with sufficient affinity for the target; a single domain antibody; an artificial single binder such as a Darpin (designed ankyrin repeat protein); or a single-chain derived from a T-cell receptor.

The antigen binding domain may comprise a domain which is not based on the antigen binding site of an antibody. For example, the antigen binding domain may comprise a domain based on a protein/peptide which is a soluble ligand for a tumour cell surface receptor (e.g. a soluble peptide such as a cytokine or a chemokine); or an extracellular domain of a membrane anchored ligand or a receptor for which the binding pair counterpart is expressed on the tumour cell. The antigen binding domain may be based on a natural ligand of the antigen.

The antigen binding domain may comprise an affinity peptide from a combinatorial library or a de novo designed affinity protein/peptide.

CARs comprise a spacer sequence to connect the antigen-binding domain with the transmembrane domain and spatially separate the antigen-binding domain from the endodomain. A flexible spacer allows the antigen-binding domain to orient in different directions to facilitate binding.

The transmembrane domain is the portion of the CAR which spans the membrane. The transmembrane domain may be any protein structure which is thermodynamically stable in a membrane. This is typically an alpha helix comprising of several hydrophobic residues. The transmembrane domain of any transmembrane protein can be used to supply the transmembrane portion of the CAR. The presence and span of a transmembrane domain of a protein can be determined by those skilled in the art using the TMHMM algorithm (http://www.cbs.dtu.dk/services/TMHMM-2.0/). Alternatively, an artificially designed TM domain may be used.

The transmembrane domain may be derived from CD28, which gives good receptor stability.

The endodomain is the signal-transmission portion of the CAR. It may be part of or associate with the intracellular domain of the CAR. After antigen recognition, receptors cluster, native CD45 and CD148 are excluded from the synapse and a signal is transmitted to the cell. The most commonly used endodomain component is that of CD3-zeta which contains 3 ITAMs. This transmits an activation signal to the T cell after antigen is bound. CD3-zeta may not provide a fully competent activation signal and additional co-stimulatory signaling may be needed. For example, chimeric CD28 and OX40 can be used with CD3-Zeta to transmit a proliferative/survival signal, or all three can be used together.

The endodomain may comprise:
(i) an ITAM-containing endodomain, such as the endodomain from CD3 zeta; and/or
(ii) a co-stimulatory domain, such as the endodomain from CD28; and/or
(iii) a domain which transmits a survival signal, for example a TNF receptor family endodomain such as OX-40 or 4-1BB.

Illustrative HCMV-specific CARs are described in Stripecke et al. (Blood. 2016; 128:5721) and Ali et al. (J. Infectious Diseases 2020; 222:853-862)—each of which is incorporated herein by reference.

Suitably, the CAR cell may be a T cell comprising an exogenous nucleic acid construct which encodes a CAR as described herein.

Methods for introducing an exogenous TCR or CAR into a cell are well known in the art and include introducing into a cell in vitro or ex vivo a polynucleotide encoding a TCR or CAR as defined herein. Suitably, the method further comprises incubating the cell under conditions causing expression of TCR or CAR. Optionally, the method may further comprise a step of purifying the engineered cells. The nucleic acid molecule encoding the TCR or CAR may be introduced into the cell using known vectors, for example a lentiviral vector. The term “vector” includes an expression vector, i.e. a construct capable of in vivo or in vitro/ex vivo expression. Also encompassed are cloning vectors.

Viral delivery systems include but are not limited to adenovirus vector, an adeno-associated viral (AAV) vector, a herpes viral vector, retroviral vector, lentiviral vector, baculoviral vector.

Retroviruses are RNA viruses with a life cycle different to that of lytic viruses. In this regard, a retrovirus is an infectious entity that replicates through a DNA intermediate. When a retrovirus infects a cell, its genome is converted to a DNA form by a reverse transcriptase enzyme. The DNA copy serves as a template for the production of new RNA genomes and virally encoded proteins necessary for the assembly of infectious viral particles.

There are many retroviruses, for example murine leukemia virus (MLV), human immunodeficiency virus (HIV), equine infectious anaemia virus (EIAV), mouse mammary tumour virus (MMTV), Rous sarcoma virus (RSV), Fujinami sarcoma virus (FuSV), Moloney murine leukemia virus (Mo-MLV), FBR murine osteosarcoma virus (FBR MSV), Moloney murine sarcoma virus (Mo-MSV), Abelson murine leukemia virus (A-MLV), Avian myelocytomatosis virus-29 (MC29), and Avian erythroblastosis virus (AEV) and all other retroviridiae including lentiviruses. A detailed list of retroviruses may be found in Coffin et al (“Retroviruses” 1997 Cold Spring Harbour Laboratory Press Eds: J M Coffin, S M Hughes, H E Varmus pp 758-763). Lentiviruses also belong to the retrovirus family, but they can infect both dividing and non-dividing cells (Lewis et al (1992) EMBO J. 3053-3058). The vector may be a retroviral vector. The vector may be based on or derivable from the MP71 vector backbone. The vector may lack a full-length or truncated version of the Woodchuck Hepatitis Response Element (WPRE). For efficient infection of human cells, viral particles may be packaged with amphotropic envelopes or gibbon ape leukemia virus envelopes.

Suitably, a TCR-gene-engineered cell or CAR cell as described herein may be treated in vitro or ex vivo with an immunogenic composition comprising a nucleic acid sequence or vector as described herein. Following in vitro or ex vivo treatment with the immunogenic composition, the TCR-gene-engineered cell or CAR cell may subsequently be administered to a subject to treat and/or prevent human herpesvirus infection as described herein.

Accordingly, in one aspect the present invention provides a method of treating and/or preventing a human herpesvirus infection in a subject which comprises the following steps:
(i) isolation of a cell-containing sample from a subject;
(ii) introducing a nucleic acid sequence encoding a TCR or CAR to the cells; and
(iii) administering the cells from (ii) to the subject.

Suitably the cells from (ii) may be expanded in vitro before administration to the subject.

The subject in steps (i) and (iii) may be the same or a different subject. In other words, the cells may be autologous or may be allogenic to the subject to be treated.

Step (ii) may optionally also comprise introducing a nucleic acid sequence or vector of the invention which is capable of expressing the human herpesvirus immunogenic peptide as described herein.

Suitable activators of innate immunity include, but are not limited to, toll-like receptor (TLR) agonists and RIG-I/NOD agonists. Examples of TLR agonists include TLR-7 agonists, TLR-8 agonists and TLR-9 agonists. These agonists are typically provided as small molecules in a composition for oral administration.

### Dosage

The skilled person can readily determine an appropriate dose of one of the agents of the invention to administer to a subject without undue experimentation. Typically, a physician will determine the actual dosage which will be most suitable for an individual patient and it will depend on a variety of factors including the activity of the specific agent employed, the metabolic stability and length of action of that agent, the age, body weight, general health, sex, diet, mode and time of administration, rate of excretion, drug combination, the severity of the particular condition, and the individual undergoing therapy. There can of course be individual instances where higher or lower dosage ranges are merited, and such are within the scope of the invention.

The skilled person appreciates, for example, that route of delivery (e.g. oral vs. intravenous vs. subcutaneous etc.) may impact the required dosage (and vice versa). For example, where particularly high concentrations of an agent within a particular site or location are desired, focussed delivery may be preferred. Other factors to be considered when optimizing routes and/or dosing schedule for a given therapeutic regimen may include, for example, the disease being treated (e.g. type or stage etc.), the clinical condition of a subject (e.g. age, overall health etc.), the presence or absence of combination therapy, and other factors known to medical practitioners.

The dosage is such that it is sufficient to improve symptoms or markers of the disease—as described herein.

### Subject

A “subject” refers to either a human or non-human animal.

Examples of non-human animals include vertebrates, for example mammals, such as non-human primates (particularly higher primates).

Preferably, the subject is a human. The subject may be any age, gender or ethnicity.

The subject may have a human herpesvirus infection, or be thought to be at risk from contracting or developing a human herpesvirus infection, because of, for example, a compromised immune system, an immature immune system, family history of the disease or the presence of genetic or phenotypic (e.g. biomarkers) associated with the disease.

The subject may show one or more signs or symptoms of a human herpesvirus infection. The subject may have been previously characterised as having a human herpesvirus infection by other diagnostic methods.

The subject may have been previously treated with antiviral therapy (e.g. GCV), CMVIG or a human herpesvirus vaccine (e.g. a gB vaccine, such as a HCMV gB vaccine).

In some embodiments, the subject is:
a) an individual awaiting a solid organ transplant;
b) a solid organ transplant recipient;
c) a woman of childbearing age;
d) a pregnant woman; and/or
e) a child.

In one embodiment, the subject is an individual awaiting a solid organ transplant.

In one embodiment, the subject is a solid organ transplant recipient.

In one embodiment, the subject is a woman of childbearing age.

In one embodiment, the subject is a pregnant woman.

In one embodiment, the subject is a child. Suitably, a child may be an individual between 12 months and 15 years of age.

### Variants, Homologues and Fragments

In addition to the specific proteins and nucleotides mentioned herein, the invention also encompasses the use of variants, derivatives, analogues, homologues and fragments thereof.

In the context of the invention, a variant of any given sequence is a sequence in which the specific sequence of residues (whether amino acid or nucleic acid residues) has been modified in such a manner that the polypeptide or polynucleotide in question substantially retains its function. A variant sequence can be obtained by addition, deletion, substitution, modification, replacement and/or variation of at least one residue present in the naturally-occurring protein.

The term “derivative” as used herein, in relation to proteins or polypeptides of the invention includes any substitution of, variation of, modification of, replacement of, deletion of and/or addition of one (or more) amino acid residues from or to the sequence providing that the resultant protein or polypeptide substantially retains at least one of its endogenous functions.

The term “analogue” as used herein, in relation to polypeptides or polynucleotides includes any mimetic, that is, a chemical compound that possesses at least one of the endogenous functions of the polypeptides or polynucleotides which it mimics.

Typically, amino acid substitutions may be made, for example from 1, 2 or 3 to 10 or 20 substitutions provided that the modified sequence substantially retains the required activity or ability. Amino acid substitutions may include the use of non-naturally occurring analogues.

Proteins used in the invention may also have deletions, insertions or substitutions of amino acid residues which produce a silent change and result in a functionally equivalent protein. Deliberate amino acid substitutions may be made on the basis of similarity in polarity, charge, solubility, hydrophobicity, hydrophilicity and/or the amphipathic nature of the residues as long as the endogenous function is retained. For example, negatively charged amino acids include aspartic acid and glutamic acid; positively charged amino acids include lysine and arginine; and amino acids with uncharged polar head groups having similar hydrophilicity values include asparagine, glutamine, serine, threonine and tyrosine.

The term “homologue” as used herein means an entity having a certain homology with the wild type amino acid sequence and the wild type nucleotide sequence. The term “homology” can be equated with “identity”.

A homologous sequence may include an amino acid sequence which may be at least 50%, 55%, 60%, 65%, 70%, 75%, 80%, 85% or 90% identical, preferably at least 95% or 97% or 99% identical to the subject sequence. Typically, the homologues will comprise the same active sites etc. as the subject amino acid sequence. Although homology can also be considered in terms of similarity (i.e. amino acid residues having similar chemical properties/functions), in the context of the invention it is preferred to express homology in terms of sequence identity.

A homologous sequence may include a nucleotide sequence which may be at least 50%, 55%, 60%, 65%, 70%, 75%, 80%, 85% or 90% identical, preferably at least 95% or 97% or 99% identical to the subject sequence. Although homology can also be considered in terms of similarity, in the context of the invention it is preferred to express homology in terms of sequence identity.

Preferably, reference to a sequence which has a percent identity to any one of the SEQ ID NOs detailed herein refers to a sequence which has the stated percent identity over the entire length of the SEQ ID NO referred to.

Homology comparisons can be conducted by eye or, more usually, with the aid of readily available sequence comparison programs. These commercially available computer programs can calculate percentage homology or identity between two or more sequences.

Percentage homology may be calculated over contiguous sequences, i.e. one sequence is aligned with the other sequence and each amino acid in one sequence is directly compared with the corresponding amino acid in the other sequence, one residue at a time. This is called an “ungapped” alignment. Typically, such ungapped alignments are performed only over a relatively short number of residues.

Although this is a very simple and consistent method, it fails to take into consideration that, for example, in an otherwise identical pair of sequences, one insertion or deletion in the nucleotide sequence may cause the following codons to be put out of alignment, thus potentially resulting in a large reduction in percent homology when a global alignment is performed. Consequently, most sequence comparison methods are designed to produce optimal alignments that take into consideration possible insertions and deletions without penalising unduly the overall homology score. This is achieved by inserting “gaps” in the sequence alignment to try to maximise local homology.

However, these more complex methods assign “gap penalties” to each gap that occurs in the alignment so that, for the same number of identical amino acids, a sequence alignment with as few gaps as possible, reflecting higher relatedness between the two compared sequences, will achieve a higher score than one with many gaps. “Affine gap costs” are typically used that charge a relatively high cost for the existence of a gap and a smaller penalty for each subsequent residue in the gap. This is the most commonly used gap scoring system. High gap penalties will of course produce optimised alignments with fewer gaps. Most alignment programs allow the gap penalties to be modified. However, it is preferred to use the default values when using such software for sequence comparisons. For example when using the GCG Wisconsin Bestfit package the default gap penalty for amino acid sequences is −12 for a gap and −4 for each extension.

Calculation of maximum percentage homology therefore firstly requires the production of an optimal alignment, taking into consideration gap penalties. A suitable computer program for carrying out such an alignment is the GCG Wisconsin Bestfit package (University of Wisconsin, U.S.A.; Devereux et al. (1984) Nucleic Acids Res. 12:387). Examples of other software that can perform sequence comparisons include, but are not limited to, the BLAST package (see Ausubel et al. (1999) ibid-Ch. 18), FASTA (Atschul et al. (1990) J. Mol. Biol. 403-410) and the GENEWORKS suite of comparison tools. Both BLAST and FASTA are available for offline and online searching (see Ausubel et al. (1999) ibid, pages 7-58 to 7-60). However, for some applications, it is preferred to use the GCG Bestfit program. Another tool, called BLAST 2 Sequences is also available for comparing protein and nucleotide sequences (see FEMS Microbiol. Lett. (1999) 174:247-50; FEMS Microbiol. Lett. (1999) 177:187-8).

Although the final percent homology can be measured in terms of identity, the alignment process itself is typically not based on an all-or-nothing pair comparison. Instead, a scaled similarity score matrix is generally used that assigns scores to each pairwise comparison based on chemical similarity or evolutionary distance. An example of such a matrix commonly used is the BLOSUM62 matrix—the default matrix for the BLAST suite of programs. GCG Wisconsin programs generally use either the public default values or a custom symbol comparison table if supplied (see the user manual for further details). For some applications, it is preferred to use the public default values for the GCG package, or in the case of other software, the default matrix, such as BLOSUM62.

Once the software has produced an optimal alignment, it is possible to calculate percent homology, preferably percent sequence identity. The software typically does this as part of the sequence comparison and generates a numerical result.

“Fragments” are also variants and the term typically refers to a selected region of the polypeptide or polynucleotide that is of interest either functionally or, for example, in an assay. “Fragment” thus refers to an amino acid or nucleic acid sequence that is a portion of a full-length polypeptide or polynucleotide.

Such variants may be prepared using standard recombinant DNA techniques such as site-directed mutagenesis. Where insertions are to be made, synthetic DNA encoding the insertion together with 5′ and 3′ flanking regions corresponding to the naturally-occurring sequence either side of the insertion site may be made. The flanking regions will contain convenient restriction sites corresponding to sites in the naturally-occurring sequence so that the sequence may be cut with the appropriate enzyme(s) and the synthetic DNA ligated into the cut. The DNA is then expressed in accordance with the invention to make the encoded protein. These methods are only illustrative of the numerous standard techniques known in the art for manipulation of DNA sequences and other known techniques may also be used.

This disclosure is not limited by the exemplary methods and materials disclosed herein, and any methods and materials similar or equivalent to those described herein can be used in the practice or testing of embodiments of this disclosure. Numeric ranges are inclusive of the numbers defining the range. Unless otherwise indicated, any nucleic acid sequences are written left to right in 5′ to 3′ orientation; amino acid sequences are written left to right in amino to carboxy orientation, respectively.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within this disclosure. The upper and lower limits of these smaller ranges may independently be included or excluded in the range, and each range where either, neither or both limits are included in the smaller ranges is also encompassed within this disclosure, subject to any specifically excluded limit in the stated range. Where the stated range includes one or both of the limits, ranges excluding either or both of those included limits are also included in this disclosure.

It must be noted that as used herein and in the appended claims, the singular forms “a”, “an”, and “the” include plural referents unless the context clearly dictates otherwise.

The terms “comprising”, “comprises” and “comprised of” as used herein are synonymous with “including”, “includes” or “containing”, “contains”, and are inclusive or open-ended and do not exclude additional, non-recited members, elements or method steps. The terms “comprising”, “comprises” and “comprised of” also include the term “consisting of”.

The publications discussed herein are provided solely for their disclosure prior to the filing date of the present application. Nothing herein is to be construed as an admission that such publications constitute prior art to the claims appended hereto.

The invention will now be further described by way of Examples, which are meant to serve to assist one of ordinary skill in the art in carrying out the invention and are not intended in any way to limit the scope of the invention.

### EXAMPLES

### Example 1

A phase II randomized controlled trial of a human cytomegalovirus (HCMV) vaccine was performed in patients awaiting a solid organ transplant. The vaccine comprised of a recombinant form of the viral protein glycoprotein B (gB) delivered with MF59 adjuvant. Volunteers were vaccinated with gB/MF59 or placebo control prior to transplant and monitored for humoral immunity to gB and, post-transplant, the development of HCMV viraemia. It was demonstrated that the partial protection observed directly correlated with the levels of anti-gB antibody response elicited in response to vaccination (Griffiths et al, 2011, Lancet, 377:1256-63).

We performed an analysis in seronegative individuals of humoral responses against known antigenic domains of gB (AD-1 to AD-5), which revealed no relationship with vaccine-mediated protection suggesting that a novel response was being induced by vaccination that could be important for protection.

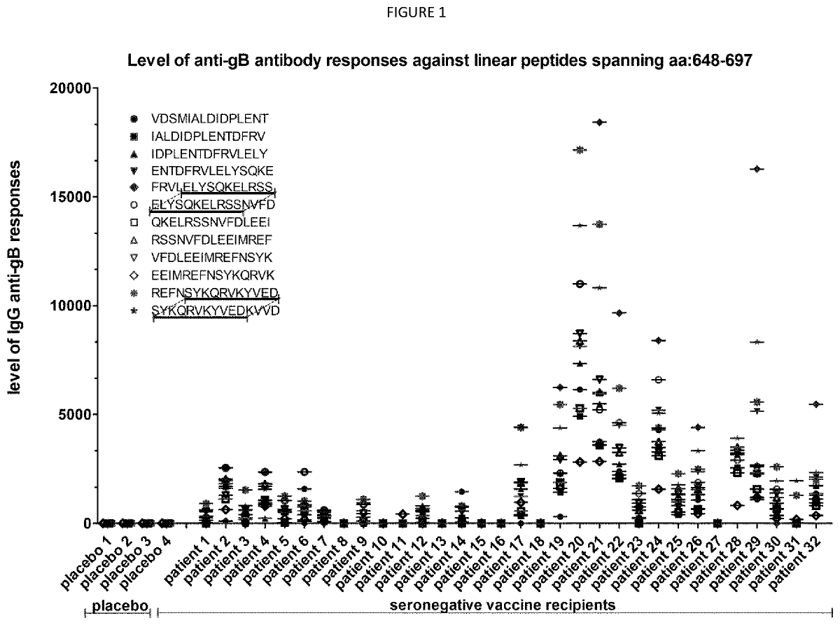

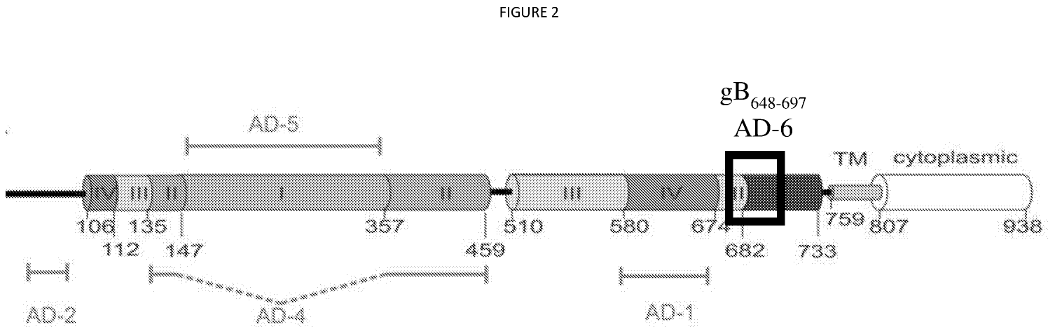

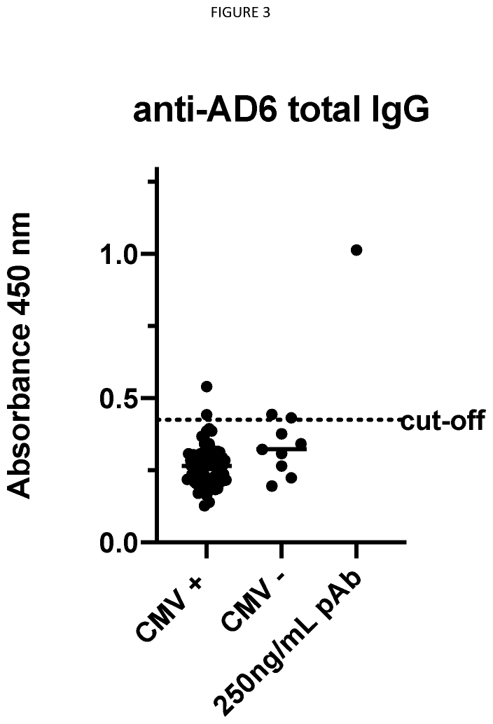

To test this, sera taken from the seronegative cohort vaccinated with gB/MF59 was analysed by gB peptide microarray (JPT Technologies). Overlapping peptides 15 amino acids in length that covered the entirety of the gB protein were used to identify novel antibody responses against linear epitopes in gB. Using this approach, strong anti-gB responses were detected in the region between amino acids 648-697 of gB (FIG. 1). These responses are against a region we are defining as AD6 (FIG. 2) and were detectable in>50% of vaccinated individuals. In contrast, this response is rarely seen in individuals naturally infected with HCMV (<5%: FIG. 3).

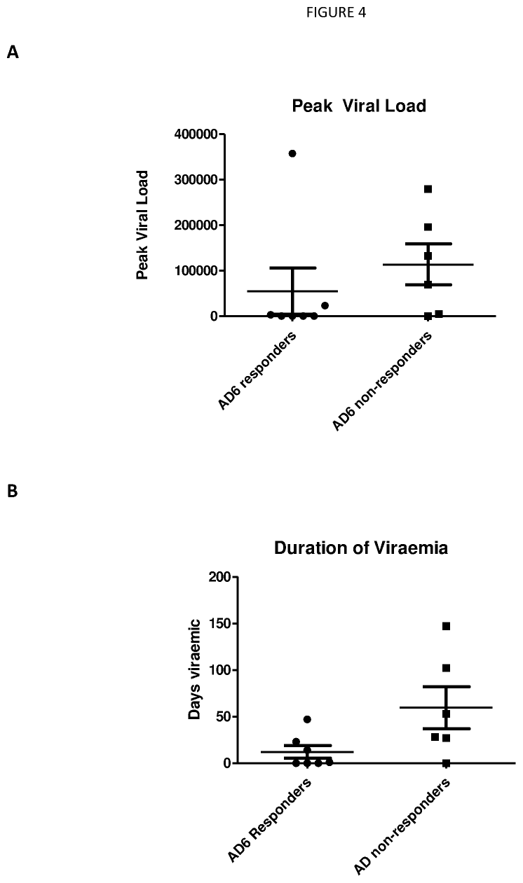

We have previously reported that antibody responses against AD1-5 do not correlate with protection in HCMV seronegative patients vaccinated with gB/MF59 (Baraniak et al, PNAS, 2018). Thus, we asked whether responses against AD6 correlated with outcome post transplant. The data show (FIG. 4) individuals with good antibody responses against AD6 post vaccination had lower peak viral loads and shorter duration of viraemia post-transplant. These data represent the first direct correlation between an epitope-specific antibody response against the gB vaccine and better outcomes post-transplant in seronegative vaccine recipients.

### Example 2

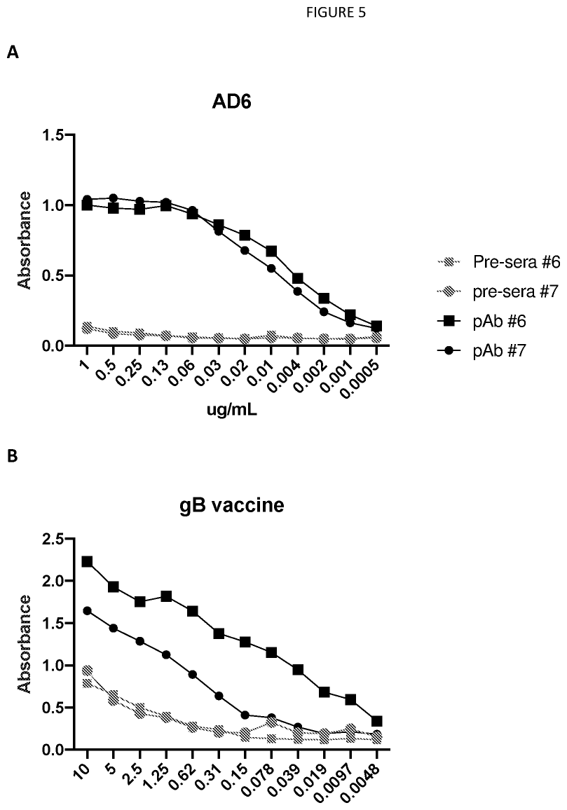

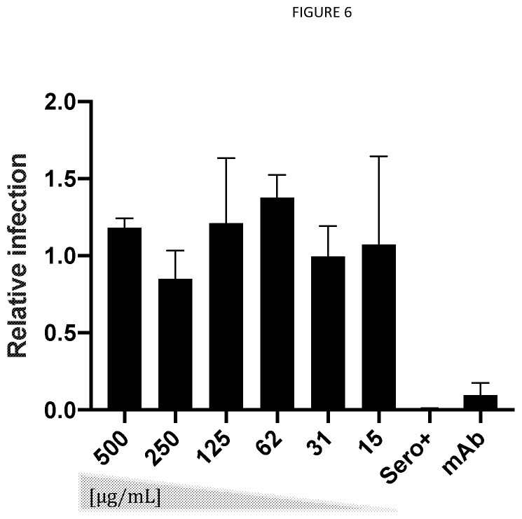

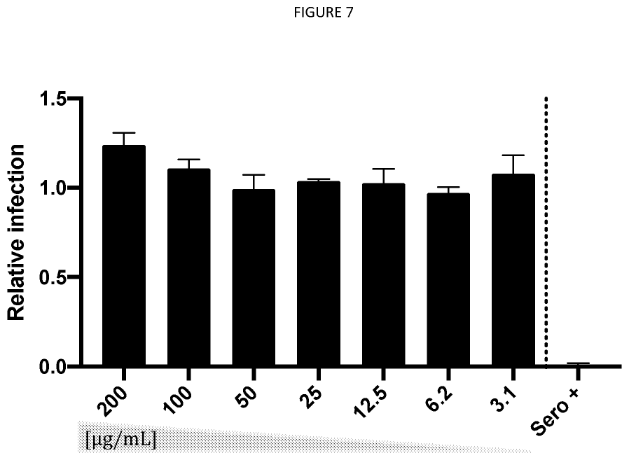

To investigate anti-AD6 antibodies in more detail, rabbits were immunized with the AD6 peptide (SEQ ID NO: 7) to generate polyclonal sera. These sera were confirmed to recognize the AD6 peptide and also the gB vaccine protein by ELISA (FIG. 5). Having established that the sera recognizes AD6 and recombinant gB we tested whether the antibody could neutralize HCMV infection. HCMV was incubated with the antibody for 1 hour prior to the infection of cells and then infection scored by staining cells for viral gene expression—a marker of infection. The data show that the rabbit AD6 antisera does not block HCMV infection of multiple cell types including fibroblasts (FIG. 6) and epithelial cells (FIG. 7). Thus, the anti-AD6 antibodies are not neutralising antibodies.

The lack of neutralizing activity observed with an AD6 antisera is consistent with our previous report that demonstrated that vaccination with gB/MF59 did not elicit potent neutralizing antibody responses against gB (Baraniak et al, PNAS, 2018).

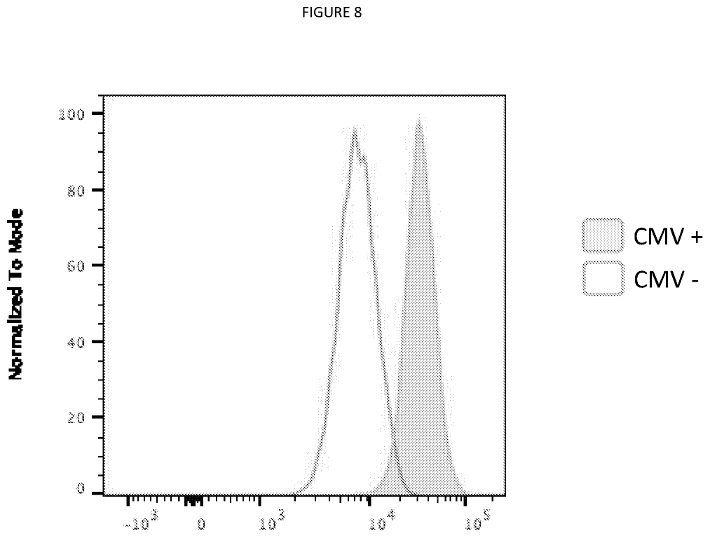

HCMV can be targeted by antibodies in two ways: via direct antibody binding to the virus (classic neutralization as measured in FIGS. 6 & 7) or via the targeting of infected cells. The gB protein is expressed on the surface of virally infected cells. Furthermore, the expression of gB on the surface of cells can be detected using the AD6 antibody. Infected cells were fixed 5 days post infection with HCMV and incubated with rabbit anti-AD6 sera and analysed by flow cytometry. Cells were infected with a strain of HCMV expressing green fluorescent protein (GFP) allowing the identification of infected (CMV+) and uninfected cells (CMV−) cells. The data show that AD6 antisera detects gB on the surface of infected cells (FIG. 8).

To test whether this had any impact on HCMV replication we utilized a strain of HCMV engineered to represent the wild type sequence (NCBI Reference Sequence: NC_006273.2) to grow predominantly as a cell associated virus in viral spreading assay. Cells were infected at a low multiplicity of infection (MOI) resulting in ˜5% infection at day 1. The cells were then cultured for 10 days to allow viral replication and spread into neighbouring cells which was enumerated using Hermes WiScan technology to establish a percentage infection in the assay.

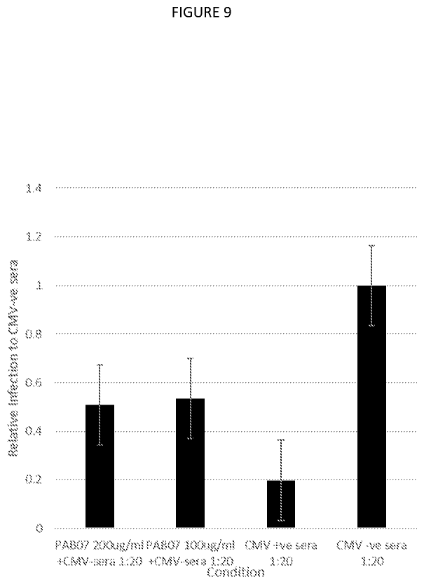

Two days post HCMV infection, cells were further incubated with rabbit anti-AD6 sera, CMV positive donor serum or CMV negative donor serum. Rate of spread of the virus in each condition was calculated relative to the CMV negative donor serum control (no CMV specific activity). Inhibition of viral spread was calculated by enumerating % infection in test wells and comparing with control conditions. A reduction in viral spread of>20% was considered functional. The data show that the spread of HCMV is reduced in the presence of the AD6 antisera compared to the serum control (FIG. 9) evidenced by a reduced percentage of infected cells at 10 days culture. Thus, the anti-AD6 sera limits the spread of HCMV in vitro.

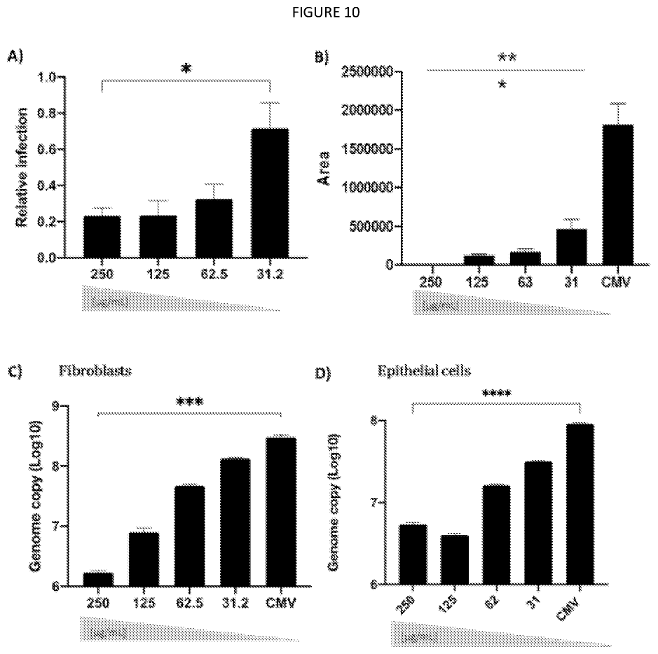

To further demonstrate that the rabbit anti-AD6 antibody blocks the spread of HCMV in vitro, we measured anti-viral control in multiple ways thereby providing a more comprehensive analysis of multiple parameters in two cell types (fibroblasts and epithelial cells). The data evidence that the anti-AD6 sera limits the spread of HCMV in vitro in both fibroblasts and epithelial cells (FIG. 10).

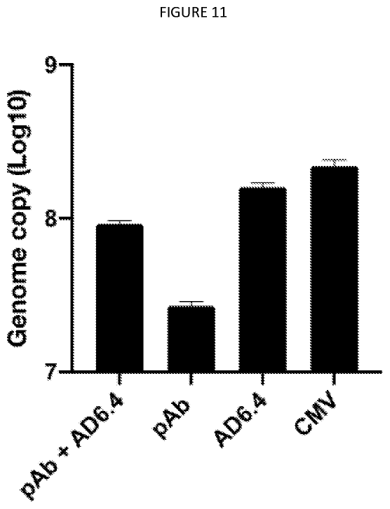

To further investigate the identified hotspots, a synthesised 15mer peptide that covers the ELYSQKELRSS peptide (denoted as AD6.4) (SEQ ID NO: 25) was pre-absorbed with anti-Ad6 antibody and the activity of the antisera tested. The data shows partial reversal of the activity of the rabbit polyclonal antisera suggesting that antibodies directed against this region are an important component of the control (FIG. 11).

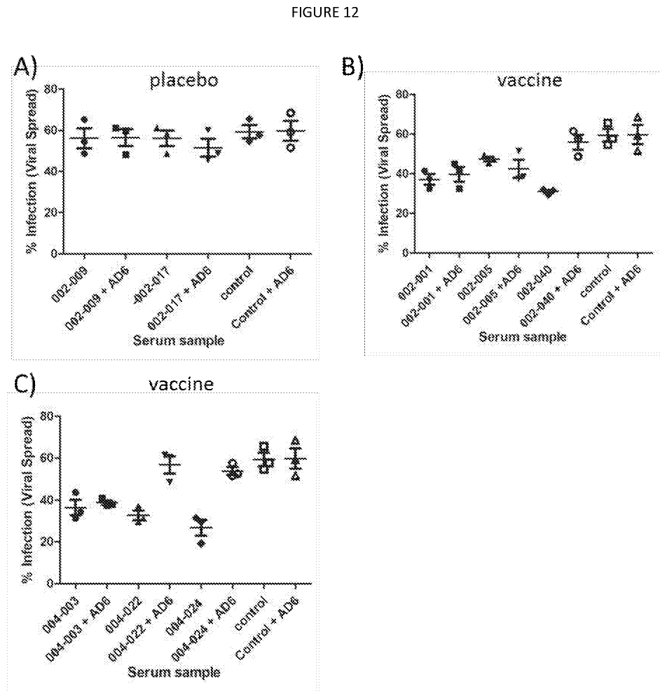

To further investigate the role of AD6 in the control of HCMV infection, we assessed the capacity of sera and sera preabsorbed with AD6 peptide to limit viral spread using human sera from HCMV seronegative individuals who received placebo or two doses of gB/MF59 vaccine. The data in FIG. 12 show the following:
A) Sera from placebo controls have no effect on spread of HCMV in vitro (this is expected as they have no HCMV specific antibodies); and
B,C) The sera from some gB/MF59 vaccine recipients can reduce the spread of HCMV in vitro and that for some patients this is blocked by preabsorbtion with AD6 protein e.g. 002-0040, 004-022, 004-024.

Overall, the data show that complete AD6 peptide (SEQ ID NO: 7) reverses the activity of human vaccine sera and its capacity to block cell spread (FIG. 12). Thus, these data provide evidence that human AD6 antibodies are active against HCMV.

All publications mentioned in the above specification are herein incorporated by reference. Various modifications and variations of the described methods and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in molecular biology or related fields are intended to be within the scope of the following claims.

1. A human herpesvirus immunogenic peptide comprising SEQ ID NO: 7, SEQ ID NO: 9 or SEQ ID NO: 11, or a sequence having at least 75% identity thereto, or an immunogenic fragment thereof.

2. The human herpesvirus immunogenic peptide according to claim 1, wherein the immunogenic peptide comprises SEQ ID NO: 7, a sequence having at least 75% identity thereto, or an immunogenic fragment thereof.

3. The human herpesvirus immunogenic peptide according to claim 1, wherein the immunogenic peptide comprises SEQ ID NO: 9, a sequence having at least 75% identity thereto, or an immunogenic fragment thereof.

4. The human herpesvirus immunogenic peptide according to claim 1, wherein the immunogenic peptide comprises SEQ ID NO: 11, a sequence having at least 75% identity thereto, or an immunogenic fragment thereof.

5. The human herpesvirus immunogenic peptide according to any one of the preceding claims, wherein the immunogenic peptide comprises a sequence having at least 80% identity to SEQ ID NO: 7, to SEQ ID NO: 9 or to SEQ ID NO: 11.

6. The human herpesvirus immunogenic peptide according to any one of the preceding claims, wherein the immunogenic fragment is about 47 or fewer amino acids in length.

7. The human herpesvirus immunogenic peptide according to any one of claim 1, 2, 5 or 6, wherein the immunogenic fragment is selected from SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25 or SEQ ID NO: 26, or a variant thereof having one, two or three amino acid substitutions and/or having one, two or three amino acid deletions at the N- and/or C-terminus.

8. The human herpesvirus immunogenic peptide according to any one of claims 1, 2 or 5 to 7, wherein the immunogenic fragment is SEQ ID NO: 25 or SEQ ID NO: 26, or a variant thereof having one, two or three amino acid substitutions and/or having one, two or three amino acid deletions at the N- and/or C-terminus.

9. The human herpesvirus immunogenic peptide according to any one of the preceding claims, wherein the human herpesvirus is human cytomegalovirus (HCMV), herpes simplex virus-1 (HSV-1), or Epstein-Barr virus (EBV).

10. The human herpesvirus immunogenic peptide according to claim 9, wherein the human herpesvirus is HCMV.

11. A nucleic acid sequence encoding the human herpesvirus immunogenic peptide according to any one of claims 1 to 10.

12. A vector comprising the nucleic acid sequence according to claim 11.

13. An inhibitor of human herpesvirus glycoprotein-B which is capable of binding to human herpesvirus glycoprotein-B on the surface of a cell.

14. The inhibitor according to claim 13, wherein the inhibitor binds within SEQ ID NO: 7, SEQ ID NO: 9 or SEQ ID NO: 11, or a sequence having at least 75% identity thereto, or a fragment thereof.

15. An inhibitor of human herpesvirus glycoprotein-B which is capable of binding to human herpesvirus glycoprotein-B, wherein the inhibitor binds within SEQ ID NO: 7, SEQ ID NO: 9 or SEQ ID NO: 11, or a sequence having at least 75% identity thereto, or a fragment thereof.

16. The inhibitor according to any one of claims 13 to 15, wherein the inhibitor binds within SEQ ID NO: 7, a sequence having at least 75% identity thereto, or an immunogenic fragment thereof.

17. The inhibitor according to any one of claims 13 to 15, wherein the inhibitor binds within SEQ ID NO: 9, a sequence having at least 75% identity thereto, or an immunogenic fragment thereof.

18. The inhibitor according to any one of claims 13 to 15, wherein the inhibitor binds within SEQ ID NO: 11, a sequence having at least 75% identity thereto, or an immunogenic fragment thereof.

19. The inhibitor according to any one of claims 13 to 16, wherein the inhibitor binds within any one of SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25 or SEQ ID NO: 26, or a variant thereof having one, two or three amino acid substitutions and/or having one, two or three amino acid deletions at the N- and/or C-terminus.

20. The inhibitor according to any one of claim 13 to 16 or 19, wherein the inhibitor binds within SEQ ID NO: 25 or SEQ ID NO: 26, or a variant thereof having one, two or three amino acid substitutions and/or having one, two or three amino acid deletions at the N- and/or C-terminus.

21. The inhibitor according to any one of claim 13 to 16, 19 or 20, wherein the inhibitor binds within a sequence having at least 80% identity to SEQ ID NO: 7, to SEQ ID NO: 9 or to SEQ ID NO: 11.

22. The inhibitor according to any one of claims 13 to 21, wherein the fragment is about 47 or fewer amino acids in length.

23. The inhibitor according to any one of claims 13 to 22, wherein the human herpesvirus is human cytomegalovirus (HCMV), herpes simplex virus-1 (HSV-1), or Epstein-Barr virus (EBV).

24. The inhibitor according to claim 23, wherein the human herpesvirus is HCMV.

25. The inhibitor according to any one of claims 13 to 24, wherein the inhibitor is selected from an antibody, an Ig fusion protein, a polypeptide, a peptide, a polynucleotide, a small molecule, a non-antibody scaffold, an aptamer, or combinations thereof.

26. The inhibitor according to claim 25, wherein the inhibitor is an antibody.

27. The inhibitor according to claim 26, wherein the antibody is a monoclonal antibody, a humanised antibody, a single-chain antibody, or an antibody fragment.

28. An immunogenic composition comprising a human herpesvirus immunogenic peptide as defined in any one of claims 1 to 10.

29. An immunogenic composition comprising a nucleic acid sequence as defined in claim 11 or a vector as defined in claim 12.

30. A pharmaceutical composition comprising an inhibitor as defined in any one of claims 13 to 27.

31. A vaccine comprising a human herpesvirus immunogenic peptide as defined in any one of claims 1 to 10.

32. A vaccine comprising a nucleic acid sequence as defined in claim 11 or a vector as defined in claim 12.

33. A vaccine comprising an inhibitor as defined in any one of claims 13 to 27.

34. An immunogenic composition comprising a human herpesvirus immunogenic peptide as defined in any one of claims 1 to 10 for use in the treatment or prevention of human herpesvirus infection in a subject.

35. An immunogenic composition comprising a nucleic acid sequence as defined in claim 11 or a vector as defined in claim 12 for use in the treatment or prevention of human herpesvirus infection in a subject.

36. A pharmaceutical composition comprising an inhibitor as defined in any one of claims 13 to 27 for use in the treatment or prevention of human herpesvirus infection in a subject.

37. A vaccine comprising a human herpesvirus immunogenic peptide as defined in any one of claims 1 to 10 for use in the treatment or prevention of human herpesvirus infection in a subject.

38. A vaccine comprising a nucleic acid sequence as defined in claim 11 or a vector as defined in claim 12 for use in the treatment or prevention of human herpesvirus infection in a subject.

39. A vaccine comprising an inhibitor as defined in any one of claims 13 to 27 for use in the treatment or prevention of human herpesvirus infection in a subject.

40. An inhibitor as defined in any one of claims 13 to 27 for use in the treatment or prevention of human herpesvirus infection in a subject.

41. A method for the treatment and/or prevention of a human herpesvirus infection, comprising administering:
a) an immunological composition comprising a human herpesvirus immunogenic peptide as defined in any one of claims 1 to 10,
b) a pharmaceutical composition according to claim 30,
c) a vaccine comprising a human herpesvirus immunogenic peptide as defined in any of claims 1 to 10, a nucleic acid sequence as defined in claim 11 or a vector as defined in claim 12, or
d) an inhibitor according to in any one of claims 13 to 27 to a subject in need thereof.

42. The immunogenic composition, pharmaceutical composition, vaccine, inhibitor for use or method according to any one of claims 34 to 41, wherein the subject is:
a) an individual awaiting a solid organ transplant;
b) a solid organ transplant recipient;
c) a woman of childbearing age;
d) a pregnant woman; or
e) a child.

43. The immunogenic composition, pharmaceutical composition, vaccine, inhibitor for use or method according to any one of claims 34 to 42, wherein the cell-to-cell spread of the human herpesvirus is inhibited.

44. The immunogenic composition, pharmaceutical composition, vaccine, inhibitor for use or method according to any one of claims 34 to 43, wherein the immunogenic composition, pharmaceutical composition, vaccine or inhibitor is for use in the prevention of human herpesvirus infection in the subject.

45. The immunogenic composition, pharmaceutical composition, vaccine, inhibitor for use or method according to any one of claims 34 to 44, wherein the human herpesvirus is human cytomegalovirus (HCMV), herpes simplex virus-1 (HSV-1), or Epstein-Barr virus (EBV).

46. The immunogenic composition, pharmaceutical composition, vaccine inhibitor for use or method according to claim 4555, wherein the human herpesvirus is HCMV.

47. The immunogenic composition, pharmaceutical composition, vaccine, inhibitor for use or method according to any one of claims 34 to 46, wherein the immunogenic composition, pharmaceutical composition, vaccine or inhibitor is administered to the subject in combination with at least one further pharmaceutically active agent.

48. The immunogenic composition, pharmaceutical composition, vaccine, inhibitor for use or method according to claim 47 wherein the at least one further pharmaceutically active agent is selected from an antiviral, a herpesvirus vaccine, an immunostimulatory cytokine, a checkpoint inhibitor, TCR-gene-engineered T cells, activators of innate immunity, monoclonal or bispecific antibodies, CAR cells, soluble T cell receptors, or any combination thereof.

49. The immunogenic composition, pharmaceutical composition, vaccine, inhibitor for use or method according to claim 47 or claim 48, wherein the immunogenic composition, vaccine or inhibitor is administered simultaneously, sequentially or separately with the at least one further pharmaceutically active agent.


SEQUENCE LISTING (28 sequences)

SEQ ID NO: 1 - protein, 906 aa, Human cytomegalovirus
MESRIWCLVVCVNLCIVCLGAAVSSSSTSHATSSTHNGSHTSRTTSAQTRSVYSQHVTSSEAVSHRANETIYNTTLKYGDVVGVNTTKYPYRVCSMAQGTDLIRFERNIICTSMKPINEDLDEGIMVVYKRNIVAHTFKVRVYQKVLTFRRSYAYIYTTYLLGSNTEYVAPPMWEIHHINKFAQCYSSYSRVIGGTVFVAYHRDSYENKTMQLIPDDYSNTHSTRYVTVKDQWHSRGSTWLYRETCNLNCMLTITTARSKYPYHFFATSTGDVVYISPFYNGTNRNASYFGENADKFFIFPNYTIVSDFGRPNAAPETHRLVAFLERADSVISWDIQDEKNVTCQLTFWEASERTIRSEAEDSYHFSSAKMTATFLSKKQEVNMSDSALDCVRDEAINKLQQIFNTSYNQTYEKYGNVSVFETSGGLVVFWQGIKQKSLVELERLANRSSLNITHRTRRSTSDNNTTHLSSMESVHNLVYAQLQFTYDTLRGYINRALAQIAEAWCVDQRRTLEVFKELSKINPSAILSAIYNKPIAARFMGDVLGLASCVTINQTSVKVLRDMNVKESPGRCYSRPVVIFNFANSSYVQYGQLGEDNEILLGNHRTEECQLPSLKIFIAGNSAYEYVDYLFKRMIDLSSISTVDSMIALDIDPLENTDFRVLELYSQKELRSSNVFDLEEIMREFNSYKQRVKYVEDKVVDPLPPYLKGLDDLMSGLGAAGKAVGVAIGAVGGAVASVVEGVATFLKNPFGAFTIILVAIAVVIITYLIYTRQRRLCTQPLQNLFPYLVSADGTTVTSGSTKDTSLQAPPSYEESVYNSGRKGPGPPSSDASTAAPPYTNEQAYQMLLALARLDAEQRAQQNGTDSLDGQTGTQDKGQKPNLLDRLRHRKNGYRHLKDSDEEENV

SEQ ID NO: 2 - DNA, 2721 bp, Human cytomegalovirus
atggaatccaggatctggtgcctggtagtctgcgttaacctgtgtatcgtctgtctgggtgctgcggtttcctcttctagtacttcccatgcaacttcttctactcacaatggaagccatacttctcgtacgacgtctgctcaaacccggtcagtctattctcaacacgtaacgtcttctgaagccgtcagtcatagagccaacgagactatctacaacactaccctcaagtacggagatgtggtgggagtcaacactaccaagtacccctatcgcgtgtgttctatggcccagggtacggatcttattcgctttgaacgtaatatcatctgcacctcgatgaagcctatcaatgaagacttggatgagggcatcatggtggtctacaagcgcaacatcgtggcgcacacctttaaggtacgggtctaccaaaaggttttgacgtttcgtcgtagctacgcttacatctacaccacttatctgctgggcagcaatacggaatacgtggcgcctcctatgtgggagattcatcacatcaacaagtttgctcaatgctacagttcctacagccgcgttataggaggcacggttttcgtggcatatcatagggacagttatgaaaacaaaaccatgcaattaattcccgacgattattccaacacccacagtacccgttacgtgacggtcaaggatcagtggcacagccgcggcagcacctggctctatcgtgagacctgtaatctgaactgtatgctgaccatcactactgcgcgctccaagtatccttatcatttttttgcaacttccacgggtgatgtggtttacatttctcctttctacaacggaaccaatcgcaatgccagctactttggagaaaacgccgacaagtttttcattttcccgaactacaccatcgtttccgactttggaagacccaacgctgcgccagaaacccataggttggtggcttttctcgaacgtgccgactcggtgatctcttgggatatacaggacgagaagaatgtcacctgccagctcaccttctgggaagcctcggaacgtactatccgttccgaagccgaagactcgtaccacttttcttctgccaaaatgactgcaacttttctgtctaagaaacaagaagtgaacatgtccgactccgcgctggactgcgtacgtgatgaggctataaataagttacagcagattttcaatacttcatacaatcaaacatatgaaaaatacggaaacgtgtccgtcttcgaaaccagcggcggtctggtggtgttctggcaaggcatcaagcaaaaatctttggtggaattggaacgtttggccaatcgatccagtctgaatatcactcataggaccagaagaagtacgagtgacaataatacaactcatttgtccagcatggaatcggtgcacaatctggtctacgcccagctgcagttcacctatgacacgttgcgcggttacatcaaccgggcgctggcgcaaatcgcagaagcctggtgtgtggatcaacggcgcaccctagaggtcttcaaggaactcagcaagatcaacccgtcagccattctctcggccatttacaacaaaccgattgccgcgcgtttcatgggtgatgtcttgggcctggccagctgcgtgaccatcaaccaaaccagcgtcaaggtgctgcgtgatatgaacgtgaaggaatcgccaggacgctgctactcacgacccgtggtcatctttaatttcgccaacagctcgtacgtgcagtacggtcaactgggcgaggacaacgaaatcctgttgggcaaccaccgcactgaggaatgtcagcttcccagcctcaagatcttcatcgccgggaactcggcctacgagtacgtggactacctcttcaaacgcatgattgacctcagcagtatctccaccgtcgacagcatgatcgccctggatatcgacccgctggaaaataccgacttcagggtactggaactttactcgcagaaagagctgcgttccagcaacgtttttgacctcgaagagatcatgcgcgaattcaactcgtacaagcagcgggtaaagtacgtggaggacaaggtagtcgacccgctaccgccctacctcaagggtctggacgacctcatgagcggcctgggcgccgcgggaaaggccgttggcgtagccattggggccgtgggtggcgcggtggcctccgtggtcgaaggcgttgccaccttcctcaaaaaccccttcggagccttcaccatcatcctcgtggccatagccgtagtcattatcacttatttgatctatactcgacagcggcgtctgtgcacgcagccgctgcagaacctctttccctatctggtgtccgccgacgggaccaccgtgacgtcgggcagcaccaaagacacgtcgttacaggctccgccttcctacgaggaaagtgtttataattctggtcgcaaaggaccgggaccaccgtcgtctgatgcatccacggcggctccgccttacaccaacgagcaggcttaccagatgcttctggccctggcccgtctggacgcagagcagcgagcgcagcagaacggtacagattctttggacggacagactggcacgcaggacaagggacagaagcctaacctgctagaccggctgcgacatcgcaaaaacggctacagacacttgaaagactccgacgaagaagagaacgtctga

SEQ ID NO: 3 - protein, 904 aa, Artificial Sequence
  illustrative HSV-1 (strain KOS) gB sequence
MHQGAPSWGRRWFVVWALLGLTLGVLVASAAPTSPGTPGVAAATQAANGGPATPAPPPLGAAPTGDPKPKKNKKPKNPTPPRPAGDNATVAAGHATLREHLRDIKAENTDANFYVCPPPTGATVVQFEQPRRCPTRPEGQNYTEGIAVVFKENIAPYKFKATMYYKDVTVSQVWFGHRYSQFMGIFEDRAPVPFEEVIDKINAKGVCRSTAKYVRNNLETTAFHRDDHETDMELKPANAATRTSRGWHTTDLKYNPSRVEAFHRYGTTVNCIVEEVDARSVYPYDEFVLATGDFVYMSPFYGYREGSHTEHTTYAADRFKQVDGFYARDLTTKARATAPTTRNLLTTPKFTVAWDWVPKRPSVCTMTKWQEVDEMLRSEYGGSFRFSSDAISTTFTTNLTEYPLSRVDLGDCIGKDARDAMDRIFARRYNATHIKVGQPQYYQANGGFLIAYQPLLSNTLAELYVREHLREQSRKPPNPTPPPPGASANASVERIKTTSSIEFARLQFTYNHIQRHVNDMLGRVAIAWCELQNHELTLWNEARKLNPNAIASVTVGRRVSARMLGDVMAVSTCVPVAADNVIVQNSMRISSRPGACYSRPLVSFRYEDQGPLVEGQLGENNELRLTRDAIEPCTVGHRRYFTFGGGYVYFEEYAYSHQLSRADITTVSTFIDLNITMLEDHEFVPLEVYTRHEIKDSGLLDYTEVQRRNQLHDLRFADIDTVIHADANAAMFAGLGAFFEGMGDLGRAVGKVVMGIVGGVVSAVSGVSSFMSNPFGALAVGLLVLAGLAAAFFAFRYVMRLQSNPMKALYPLTTKELKNPTNPDASGEGEEGGDFDEAKLAEAREMIRYMALVSAMERTEHKAKKKGTSALLSAKVTDMVMRKRRNTNYTQVPNKDGDADEDDL

SEQ ID NO: 4 - DNA, 2715 bp, Artificial Sequence
  illustrative HSV-1 (strain KOS) gB sequence
atgcaccagggcgccccctcgtgggggcgccggtggttcgtcgtatgggcgctcttggggttgacgctgggggtcctggtggcgtcggcggctccgacttcccccggcacgcctggggtcgcggccgcgacccaggcggcgaacgggggccctgccactccggcgccgccgccccttggcgccgccccaacgggggacccgaaaccgaagaagaacaaaaaaccgaaaaacccaacgccaccacgccccgccggcgacaacgcgaccgtcgccgcgggccacgccaccctgcgcgagcacctgcgggacatcaaggcggagaacaccgatgcaaacttttacgtgtgcccaccccccacgggcgccacggtggtgcagttcgagcagccgcgccgctgcccgacccggcccgagggtcagaactacacggagggcatcgcggtggtcttcaaggagaacatcgccccgtacaagttcaaggccaccatgtactacaaagacgtcaccgtttcgcaggtgtggttcggccaccgctactcccagtttatggggatctttgaggaccgcgcccccgtccccttcgaggaggtgatcgacaagatcaacgccaagggggtctgtcggtccacggccaagtacgtgcgcaacaacctggagaccaccgcgtttcaccgggacgaccacgagaccgacatggagctgaaaccggccaacgccgcgacccgcacgagccggggctggcacaccaccgacctcaagtacaacccctcgcgggtggaggcgttccaccggtacgggacgacggtaaactgcatcgtcgaggaggtggacgcgcgctcggtgtacccgtacgacgagtttgtgctggcgactggcgactttgtgtacatgtccccgttttacggctaccgggaggggtcgcacaccgaacacaccacgtacgccgccgaccgcttcaagcaggtcgacggcttctacgcgcgcgacctcaccaccaaggcccgggccacggcgccgaccacccggaacctgctcacgacccccaagttcaccgtggcctgggactgggtgccaaagcgcccgtcggtctgcaccatgaccaagtggcaggaagtggacgagatgctgcgctccgagtacggcggctccttccgattctcctccgacgccatatccaccaccttcaccaccaacctgaccgagtacccgctctcgcgcgtggacctgggggactgcatcggcaaggacgcccgcgacgccatggaccgcatcttcgcccgcaggtacaacgcgacgcacatcaaggtgggccagccgcagtactaccaggccaatgggggctttctgatcgcgtaccagccccttctcagcaacacgctcgcggagctgtacgtgcgggaacacctccgagagcagagccgcaagcccccaaaccccacgcccccgccgcccggggccagcgccaacgcgtccgtggagcgcatcaagaccacctcctccatcgagttcgcccggctgcagtttacgtacaaccacatacagcgccatgtcaacgatatgttgggccgcgttgccatcgcgtggtgcgagctacagaatcacgagctgaccctgtggaacgaggcccgcaagctgaaccccaacgccatcgcctcggtcaccgtgggccggcgggtgagcgcgcggatgctcggcgacgtgatggccgtctccacgtgcgtgccggtcgccgcggacaacgtgatcgtccaaaactcgatgcgcatcagctcgcggcccggggcctgctacagccgccccctggtcagctttcggtacgaagaccagggcccgttggtcgaggggcagctgggggagaacaacgagctgcggctgacgcgcgatgcgatcgagccgtgcaccgtgggacaccggcgctacttcaccttcggtgggggctacgtgtacttcgaggagtacgcgtactcccaccagctgagccgcgccgacatcaccaccgtcagcaccttcatcgacctcaacatcaccatgctggaggatcacgagtttgtccccctggaggtgtacacccgccacgagatcaaggacagcggcctgctggactacacggaggtccagcgccgcaaccagctgcacgacctgcgcttcgccgacatcgacacggtcatccacgccgacgccaacgccgccatgttcgcgggcctgggcgcgttcttcgaggggatgggcgacctggggcgcgcggtcggcaaggtggtgatgggcatcgtgggcggcgtggtatcggccgtgtcgggcgtgtcctccttcatgtccaacccctttggggcgctggccgtgggtctgttggtcctggccggcctggcggcggccttcttcgcctttcgttacgtcatgcggctgcagagcaaccccatgaaggccctgtaccctctaaccaccaaggagctcaagaaccccaccaacccggacgcgtccggggagggcgaggagggcggcgactttgacgaggccaagctagccgaggccagggagatgatacggtacatggccctggtgtcggccatggagcgcacggaacacaaggccaagaagaagggcacgagcgcgctgctcagcgccaaggtcaccgacatggtcatgcgcaagcgccgcaacaccaactacacccaagttcccaacaaagacggtgacgccgacgaggacgacctgtga

SEQ ID NO: 5 - protein, 857 aa, Artificial Sequence
  illustrative EBV (strain B958) gB sequence
MTRRRVLSVVVLLAALACRLGAQTPEQPAPPATTVQPTATRQQTSFPFRVCELSSHGDLFRFSSDIQCPSFGTRENHTEGLLMVFKDNIIPYSFKVRSYTKIVTNILIYNGWYADSVTNRHEEKFSVDSYETDQMDTIYQCYNAVKMTKDGLTRVYVDRDGVNITVNLKPTGGLANGVRRYASQTELYDAPGWLIWTYRTRTTVNCLITDMMAKSNSPFDFFVTTTGQTVEMSPFYDGKNKETFHERADSFHVRTNYKIVDYDNRGTNPQGERRAFLDKGTYTLSWKLENRTAYCPLQHWQTFDSTIATETGKSIHFVTDEGTSSFVTNTTVGIELPDAFKCIEEQVNKTMHEKYEAVQDRYTKGQEAITYFITSGGLLLAWLPLTPRSLATVKNLTELTTPTSSPPSSPSPPAPSAARGSTPAAVLRRRRRDAGNATTPVPPTAPGKSLGTLNNPATVQIQFAYDSLRRQINRMLGDLARAWCLEQKRQNMVLRELTKINPTTVMSSIYGKAVAAKRLGDVISVSQCVPVNQATVTLRKSMRVPGSETMCYSRPLVSFSFINDTKTYEGQLGTDNEIFLTKKMTEVCQATSQYYFQSGNEIHVYNDYHHFKTIELDGIATLQTFISLNTSLIENIDFASLELYSRDEQRASNVFDLEGIFREYNFQAQNIAGLRKDLDNAVSNGRNQFVDGLGELMDSLGSVGQSITNLVSTVGGLFSSLVSGFISFFKNPFGGMLILVLVAGVVILVISLTRRTRQMSQQPVQMLYPGIDELAQQHASGEGPGINPISKTELQAIMLALHEQNQEQKRAAQRAAGPSVASRALQAARDRFPGLRRRRYHDPETAAALLGEAETEF

SEQ ID NO: 6 - DNA, 2573 bp, Artificial Sequence
  illustrative EBV (strain B958) gB sequence
atgactcggcgtagggtgctaagcgtggtcgtgctgctagccgccctggcgtgccgtctcggtgcgcagaccccagagcagcccgcaccccccgccaccacggtgcagcctaccgccacgcgtcagcaaaccagctttcctttccgagtctgcgagctctccagccacggcgacctgttccgcttctcctcggacatccagtgtccctcgtttggcacgcgggagaatcacacggagggcctgttgatggtgtttaaagacaacattattccctactcgtttaaggtccgctcctacaccaagatagtgaccaacattctcatctacaatggctggtacgcggactccgtgaccaaccggcacgaggagaagttctccgttgacagctacgaaactgaccagatggataccatctaccagtgctacaacgcggtcaagatgacaaaagatgggctgacgcgcgtgtatgtagaccgcgacggagttaacatcaccgtcaacctaaagcccaccgggggcctggccaacggggtgcgccgctacgccagccagacggagctctatgacgcccccgggtggttgatatggacttacagaacaagaactaccgtcaactgcctgataactgacatgatggccaagtccaacagccccttcgacttctttgtgaccaccaccgggcagactgtggaaatgtcccctttctatgacgggaaaaataaggaaaccttccatgagcgggcagactccttccacgtgagaactaactacaagatagtggactacgacaaccgagggacgaacccgcaaggcgaacgccgagccttcctggacaagggcacttacacgctatcttggaagctcgagaacaggacagcctactgcccgcttcaacactggcaaacctttgactcgaccatcgccacagaaacagggaagtcaatacattttgtgactgacgagggcacctctagcttcgtgaccaacacaaccgtgggcatagagctcccggacgccttcaagtgcatcgaagagcaggtgaacaagaccatgcatgagaagtacgaggccgtccaggatcgttacacgaagggccaggaagccattacatattttataacgagcggaggattgttattagcttggctacctctgaccccgcgctcgttggccaccgtcaagaacctgacggagcttaccactccgacttcctcaccccccagcagtccatcgcccccagccccatccgcggcccgcgggagcacccccgccgccgttctgaggcgtcggaggcgggatgcggggaacgccaccacaccggtgccccccacggcccccgggaagtccctgggcaccctcaacaatcccgccaccgtccagatccaatttgcctacgactccctgcgccgccagatcaaccgcatgctgggagaccttgcgcgggcctggtgcctggagcagaagaggcagaacatggtgctgagagaactaaccaagattaatccaaccaccgtcatgtccagcatctacggtaaggcggtggcggccaagcgcctgggggatgtcatctcagtctcccagtgcgtgcccgttaaccaggccaccgtcaccctgcgcaagagcatgagggtccctggctccgagaccatgtgctactcgcgccccctggtgtccttcagctttatcaacgacaccaagacctacgagggacagctgggcaccgacaacgagatcttcctcacaaaaaagatgacggaggtgtgccaggcgaccagccagtactacttccagtccggcaacgagatccacgtctacaacgactaccaccactttaaaaccatcgagctggacggcattgccaccctgcagaccttcatctcactaaacacctccctcatcgagaacattgactttgcctccctggagctgtactcacgggacgaacagcgtgcctccaacgtctttgacctggagggcatcttccgggagtacaacttccaggcgcaaaacatcgccggcctgcggaaggatttggacaatgcagtgtcaaacggaagaaatcaattcgtggacggcctgggggaacttatggacagtctgggtagcgtgggtcagtccatcaccaacctagtcagcacggtggggggtttgtttagcagcctggtctctggtttcatctccttcttcaaaaaccccttcggcggcatgctcattctggtcctggtggcgggggtggtgatcctggttatttccctcacgaggcgcacgcgccagatgtcgcagcagccggtgcagatgctctaccccgggatcgacgagctcgctcagcaacatgcctctggtgagggtccaggcattaatcccattagtaagacagaattacaagccatcatgttagcgctgcatgagcaaaaccaggagcaaaagagagcagctcagagggcggccggaccctcagtggccagcagagcattgcaggcagccagggaccgttttccaggcctacgcagaagacgctatcacgatccagagaccgccgccgcactgcttggggaggcagagactgagtttta

SEQ ID NO: 7 - protein, 48 aa, Artificial Sequence
  illustrative HCMV gB AD-6 sequence
MIALDIDPLENTDFRVLELYSQKELRSSNVFDLEEIMREFNSYKQRVK

SEQ ID NO: 8 - DNA, 144 bp, Artificial Sequence
  illustrative HCMV gB AD-6 sequence
atgatcgccctggatatcgacccgctggaaaataccgacttcagggtactggaactttactcgcagaaagagctgcgttccagcaacgtttttgacctcgaagagatcatgcgcgaattcaactcgtacaagcagcgggtaaag

SEQ ID NO: 9 - protein, 48 aa, Artificial Sequence
  illustrative HSV-1 gB AD-6 sequence
FIDLNITMLEDHEFVPLEVYTRHEIKDSGLLDYTEVQRRNQLHDLRFA

SEQ ID NO: 10 - DNA, 144 bp, Artificial Sequence
  illustrative HSV-1 gB AD-6 sequence
ttcatcgacctcaacatcaccatgctggaggatcacgagtttgtccccctggaggtgtacacccgccacgagatcaaggacagcggcctgctggactacacggaggtccagcgccgcaaccagctgcacgacctgcgcttcgcc

SEQ ID NO: 11 - protein, 48 aa, Artificial Sequence
  illustrative EBV gB AD-6 sequence
FISLNTSLIENIDFASLELYSRDEQRASNVFDLEGIFREYNFQAQNIA

SEQ ID NO: 12 - DNA, 144 bp, Artificial Sequence
  illustrative EBV gB AD-6 sequence
ttcatctcactaaacacctccctcatcgagaacattgactttgcctccctggagctgtactcacgggacgaacagcgtgcctccaacgtctttgacctggagggcatcttccgggagtacaacttccaggcgcaaaacatcgcc

SEQ ID NO: 13 - protein, 15 aa, Artificial Sequence
  Illustrative immunogenic fragment of HCMV gB AD-6
VDSMIALDIDPLENT

SEQ ID NO: 14 - protein, 15 aa, Artificial Sequence
  Illustrative immunogenic fragment of HCMV gB AD-6
IALDIDPLENTDFRV

SEQ ID NO: 15 - protein, 15 aa, Artificial Sequence
  Illustrative immunogenic fragment of HCMV gB AD-6
IDPLENTDFRVLELY

SEQ ID NO: 16 - protein, 15 aa, Artificial Sequence
  Illustrative immunogenic fragment of HCMV gB AD-6
ENTDFRVLELYSQKE

SEQ ID NO: 17 - protein, 15 aa, Artificial Sequence
  Illustrative immunogenic fragment of HCMV gB AD-6
FRVLELYSQKELRSS

SEQ ID NO: 18 - protein, 15 aa, Artificial Sequence
  Illustrative immunogenic fragment of HCMV gB AD-6
ELYSQKELRSSNVFD

SEQ ID NO: 19 - protein, 15 aa, Artificial Sequence
  Illustrative immunogenic fragment of HCMV gB AD-6
QKELRSSNVFDLEEI

SEQ ID NO: 20 - protein, 15 aa, Artificial Sequence
  Illustrative immunogenic fragment of HCMV gB AD-6
RSSNVFDLEEIMREF

SEQ ID NO: 21 - protein, 15 aa, Artificial Sequence
  Illustrative immunogenic fragment of HCMV gB AD-6
VFDLEEIMREFNSYK

SEQ ID NO: 22 - protein, 15 aa, Artificial Sequence
  Illustrative immunogenic fragment of HCMV gB AD-6
EEIMREFNSYKQRVK

SEQ ID NO: 23 - protein, 15 aa, Artificial Sequence
  Illustrative immunogenic fragment of HCMV gB AD-6
REFNSYKQRVKYVED

SEQ ID NO: 24 - protein, 15 aa, Artificial Sequence
  Illustrative immunogenic fragment of HCMV gB AD-6
SYKQRVKYVEDKVVD

SEQ ID NO: 25 - protein, 11 aa, Artificial Sequence
  Illustrative immunogenic fragment of HCMV gB AD-6
ELYSQKELRSS

SEQ ID NO: 26 - protein, 11 aa, Artificial Sequence
  Illustrative immunogenic fragment of HCMV gB AD-6
SYKQRVKYVED

SEQ ID NO: 27 - protein, 907 aa, Human cytomegalovirus
MESRIWCLVVCVNLCIVCLGAAVSSSSTRGTSATHSHHSSHTTSAAHSRSGSVSQRVTSSQTVSHGVNETIYNTTLKYGDVVGVNTTKYPYRVCSMAQGTDLIRFERNIVCTSMKPINEDLDEGIMVVYKRNIVAHTFKVRVYQKVLTFRRSYAYIHTTYLLGSNTEYVAPPMWEIHHINSHSQCYSSYSRVIAGTVFVAYHRDSYENKTMQLMPDDYSNTHSTRYVTVKDQWHSRGSTWLYRETCNLNCMVTITTARSKYPYHFFATSTGDVVDISPFYNGTNRNASYFGENADKFFIFPNYTIVSDFGRPNSALETHRLVAFLERADSVISWDIQDEKNVTCQLTFWEASERTIRSEAEDSYHFSSAKMTATFLSKKQEVNMSDSALDCVRDEAINKLQQIFNTSYNQTYEKYGNVSVFETTGGLVVFWQGIKQKSLVELERLANRSSLNLTHNRTKRSTDGNNATHLSNMESVHNLVYAQLQFTYDTLRGYINRALAQIAEAWCVDQRRTLEVFKELSKINPSAILSAIYNKPIAARFMGDVLGLASCVTINQTSVKVLRDMNVKESPGRCYSRPVVIFNFANSSYVQYGQLGEDNEILLGNHRTEECQLPSLKIFIAGNSAYEYVDYLFKRMIDLSSISTVDSMIALDIDPLENTDFRVLELYSQKELRSSNVFDLEEIMREFNSYKQRVKYVEDKVVDPLPPYLKGLDDLMSGLGAAGKAVGVAIGAVGGAVASVVEGVATFLKNPFGAFTIILVAIAVVIITYLIYTRQRRLCTQPLQNLFPYLVSADGTTVTSGSTKDTSLQAPPSYEESVYNSGRKGPGPPSSDASTAAPPYTNEQAYQMLLALARLDAEQRAQQNGTDSLDGRTGTQDKGQKPNLLDRLRHRKNGYRHLKDSDEEENV

SEQ ID NO: 28 - DNA, 2724 bp, Human cytomegalovirus
atggaatccaggatctggtgcctggtagtctgcgttaacttgtgtatcgtctgtctgggtgctgcggtttcctcatcttctactcgtggaacttctgctactcacagtcaccattcctctcatacgacgtctgctgctcactctcgatccggttcagtctctcaacgcgtaacttcttcccaaacggtcagccatggtgttaacgagaccatctacaacactaccctcaagtacggagatgtggtgggggtcaataccaccaagtacccctatcgcgtgtgttctatggcccagggtacggatcttattcgctttgaacgtaatatcgtctgcacctcgatgaagcccatcaatgaagacctggacgagggcatcatggtggtctacaaacgcaacatcgtcgcgcacacctttaaggtacgagtctaccagaaggttttgacgtttcgtcgtagctacgcttacatccacaccacttatctgctgggcagcaacacggaatacgtggcgcctcctatgtgggagattcatcatatcaacagccacagtcagtgctacagttcctacagccgcgttatagcaggcacggttttcgtggcttatcatagggacagctatgaaaacaaaaccatgcaattaatgcccgacgattattccaacacccacagtacccgttacgtgacggtcaaggatcaatggcacagccgcggcagcacctggctctatcgtgagacctgtaatctgaattgtatggtgaccatcactactgcgcgctccaaatatccttatcattttttcgccacttccacgggtgacgtggttgacatttctcctttctacaacggaaccaatcgcaatgccagctactttggagaaaacgccgacaagtttttcatttttccgaactacactatcgtctccgactttggaagaccgaattctgcgttagagacccacaggttggtggcttttcttgaacgtgcggactcggtgatctcctgggatatacaggacgaaaagaatgtcacttgtcaactcactttctgggaagcctcggaacgcaccattcgttccgaagccgaggactcgtatcacttttcttctgccaaaatgaccgccactttcttatctaagaagcaagaggtgaacatgtccgactctgcgctggactgcgtacgtgatgaggctataaataagttacagcagattttcaatacttcatacaatcaaacatatgaaaaatatggaaacgtgtccgtctttgaaaccactggtggtttggtagtgttctggcaaggtatcaagcaaaaatctctggtggaactcgaacgtttggccaaccgctccagtctgaatcttactcataatagaaccaaaagaagtacagatggcaacaatgcaactcatttatccaacatggaatcggtgcacaatctggtctacgcccagctgcagttcacctatgacacgttgcgcggttacatcaaccgggcgctggcgcaaatcgcagaagcctggtgtgtggatcaacggcgcaccctagaggtcttcaaggaactcagcaagatcaacccgtcagccattctctcggccatttacaacaaaccgattgccgcgcgtttcatgggtgatgtcttgggcctggccagctgcgtgaccatcaaccaaaccagcgtcaaggtgctgcgtgatatgaacgtgaaggagtcgccaggacgctgctactcacgacccgtggtcatctttaatttcgccaacagctcgtacgtgcagtacggtcaactgggcgaggacaacgaaatcctgttgggcaaccaccgcactgaggaatgtcagcttcccagcctcaagatcttcatcgccgggaactcggcctacgagtacgtggactacctcttcaaacgcatgattgacctcagcagtatctccaccgtcgacagcatgatcgccctggatatcgacccgctggaaaataccgacttcagggtactggaactttactcgcagaaagagctgcgttccagcaacgtttttgacctcgaagagatcatgcgcgaattcaactcgtacaagcagcgggtaaagtacgtggaggacaaggtagtcgacccgctaccgccctacctcaagggtctggacgacctcatgagcggcctgggcgccgcgggaaaggccgttggcgtagccattggggccgtgggtggcgcggtggcctccgtggtcgaaggcgttgccaccttcctcaaaaaccccttcggagcgttcaccatcatcctcgtggccatagctgtagtcattatcacttatttgatctatactcgacagcggcgtttgtgcacgcagccgctgcagaacctctttccctatctggtgtccgccgacgggaccaccgtgacgtcgggcagcaccaaagacacgtcgttacaggctccgccttcctacgaggaaagtgtttataattctggtcgcaaaggaccgggaccaccgtcgtctgatgcatccacggcggctccgccttacaccaacgagcaggcttaccagatgcttctggccctggcccgtctggacgcagagcagcgagcgcagcagaacggtacagattctttggacggacggactggcacgcaggacaagggacagaagcccaacctactagaccgactgcgacatcgcaaaaacggctaccgacacttgaaagactctgacgaagaagagaacgtctga